(19) United States
(12) Patent Application Publication
(10) Pub. No.: US 2005/0267020 A1
(43) Pub. Date: Dec. 1, 2005

(54) Polypeptides derived from inducible hsp70 and pharmaceutical compositions containing the same

(76) Inventors: Olivier Faure, Laguna Niguel, CA (US); Konstadinos Kosmatopoulos, Paris (FR)

Correspondence Address: YOUNG & THOMPSON, 745 SOUTH 23RD STREET, 2ND FLOOR, ARLINGTON, VA, 22202, US

(21) Appl. No.: 10/491,096

(22) PCT Filed: Sep. 26, 2002
(86) PCT No.: PCT/EP02/10821

§ 371 Date: Jan. 3, 2005

(30) Foreign Application Priority Data
Sep. 27, 2001 (EP) 01402496.2

(51) Int. Cl. A61K 38/17; C07K 14/82

(52) U.S. Cl. 514/12; 530/350

(57) ABSTRACT

A peptide comprising an amino acid sequence of at least 8 contiguous amino acids, the amino acid sequence being at least 65% identical to a portion of the same length within the amino acid sequence of inducible Hsp70, characterized in that the peptide amino acid sequence differs from any amino acid sequence of the same length of the constitutive Hsc70 amino acid sequence by at least one amino acid, and that the peptide is able to induce in vitro or in vivo cytotoxic T lymphocytes able to specifically recognize cells naturally producing inducible Hsp70, the cytotoxic T lymphocytes being able to recognize bath mutated and non mutated Hsp70 epitopes but not Hsc70 epitopes

The present invention relates to polypeptides derived from inducible Hsp70 and pharmaceutical compositions containing the same.

Immunotherapy of cancer is a strategy which has already proven to be efficient in cancer patients (1-4). Thus, multiple tumor antigens and the corresponding epitopes recognized by cytotoxic T lymphocytes (CTL) have been recently identified, some of them being tested in cancer clinical trials. However, most of the known candidate target antigens have a very restricted expression, which is often limited to melanoma. To date, few tumor antigens expressed in a broad panel of tumors have been discovered.

The major stress-inducible heat shock protein, Hsp70, is a chaperone protein well-known as a specialized carrier of antigenic peptides (5, 6). Indeed, numerous studies demonstrated that antigenic peptides/Hsp complexes induce CD8+ responses specific for the peptides (7). These results showed that Hsp70 itself was not acting as an antigen in this system, but was rather functioning as a vehicle for antigenic peptides. Interestingly, however, the inducible Hsp70 is abundantly and preferentially expressed in human tumors, among which lung, breast, renal, ovarian carcinoma, osteosarcoma and colorectal cancer (8-16). Hsp70 over-expression in tumors can also be a consequence of hypoxia (24). Owing to the ability of Hsp70 to protect cells from a wide range of apoptotic, necrotic and hypoxic stimuli, it has been assumed that Hsp70 may confer survival advantage to tumor cells (17). Indeed, recent data indicate that Hsp70 over-expression is a general and necessary event for the survival of a majority of tumor cells (18, 19). It has been reported a privileged development of tumors with high expression of Hsp70 under various chemotherapy treatments (22, 23). There is a considerable interest in overcoming such a resistance mechanism.

Recently, Trieb et al. isolated a tumor infiltrating lymphocyte line from osteosarcoma patients which proliferated in response to the Hsp70 protein (20). Also, Gaudin et al. isolated and described a CTL clone specific for an epitope derived from a mutated sequence of Hsp70 (21). However, this clone does not show recognition of the endogenous non-mutated cognate epitope in tumor cells.

The present invention provides peptides able to induce cytotoxic T lymphocytes specifically recognizing cells naturally producing Hsp70, and particularly tumor cells. Peptides according to the invention are useful in a large variety of cancers. Virally infected cells also show an increased expression of Hsp70.

The present invention relates to peptides comprising an amino acid sequence of at least 8 contiguous amino acids, said amino acid sequence being at least 65% identical to a portion of the same length within the amino acid sequence of inducible Hsp70 and differing from any amino acid sequence of the same length of the constitutive Hsc70 amino acid sequence by at least one amino acid, said peptide being able to induce in vitro or in vivo cytotoxic T lymphocytes able to specifically recognize cells naturally producing inducible Hsp70, said cytotoxic T lymphocytes being able to recognize both mutated and non mutated Hsp70 epitopes but not Hsc70 epitopes.

In a particular embodiment, a peptide according to the invention comprises an amino acid sequence which is at least 75% identical to a portion of the same length within the amino acid sequence of inducible Hsp70.

Reverse immunology approach enables to identify and use peptide epitopes which are not necessarily immuno-dominant, and therefore would not be immunogenic in the whole protein. However, such sub-dominant epitopes can be useful and efficient to induce tumor recognition by specific lymphocytes. Also, defined 8- to 10-mer peptides act as tumor antigens only and do not keep other functional activities of the protein, for instance its peptide carrier capacity or the chaperoning function.

In search for an optimal Hsp70 vaccine candidate with a broad-spectrum efficiency, the present inventors have demonstrated that native Hsp70 is a tumor antigen recognized by CTL. Using the reverse immunology approach, the inventors have identified native epitopes with surprisingly potent immunogenicity. These epitopes can be used in vivo as such or linked to a carrier.

By “amino acid sequence of inducible Hsp70” is meant the amino acid sequence of Hsp70-1 protein having the accession number P08107 in the Swissprot bank database and encoded by Hsp70-1 or by Hsp70-2 genes. By “amino acid sequence of constitutive Hsc70” is meant the amino acid sequence of Hsc70 protein having the accession number P11142 according to Swissprot bank database. Said amino acid sequences are represented in FIG. 1, in example 1.

By “cells naturally producing inducible Hsp-70” are meant cells that express Hsp-70 in vivo, and therefore may present some Hsp70 epitopes, such as some tumoral cells or virally infected cells. This definition does not include cells that have been submitted to some modification intended to induce the expression of Hsp70, such as for example transfection by genetic material encoding for Hsp-70, or cells “pulsed” with peptides corresponding to fragments of Hsp70.

Cytotoxic T lymphocytes able to specifically recognize cells naturally producing inducible Hsp70 are characterized by various methods known by any man of the art. These methods measure cytotoxic T lymphocytes IFN-γ or TNF-α secretion, or specific lysis of peptide pulsed radiolabelled cellular targets mediated by lymphocytes. As an example, ELISPOT assay may be used.

By “T lymphocytes recognize either mutated and non mutated Hsp70 epitopes but not Hsc70 epitopes” is meant that T lymphocytes do not recognize cells expressing only epitopes from Hsc70, but do recognize cells expressing epitopes from Hsp70, and also cells expressing not only epitopes from Hsp70 but other epitopes.

The stress-inducible Hsp70 is a member of the very conserved Hsp70 family of proteins, and thus is highly homologous in sequence to other members of this family, among which the constitutive heat shock protein Hsc70.

Epitopes shared between Hsp70 and other proteins with an ubiquitous expression profile such as Hsc70 must belong to the immunological self, and must have tolerized their specific CD8+ repertoires. Therefore, in order to minimize the risk of tolerization, the specific screening of high affinity sequences is restricted to inducible Hsp70 peptides not shared with Hsc70 (Example 1).

The novel immunogenic peptides of the invention were selected on the basis of stability and high affinity for HLA class I molecules (see Example 2). Affinity is defined as the necessary peptide dose to stabilize a reference quantity of HLA class-I molecules at the cells' surface. Stability of peptide/HLA molecule complexes is measured by determination of the half life of the complexes.

In another particular embodiment, a peptide according to the invention comprises an amino acid sequence identical to a portion of the same length within the amino acid sequence of inducible Hsp-70.

In a particular embodiment, a peptide according to the invention comprises an amino acid sequence differing from any amino acid sequence of the same length of the constitutive Hsc70 amino acid sequence by at least one amino acid located from the third to the last C-terminal amino acid of the peptide. In another particular embodiment, a peptide according to the invention comprises an amino acid sequence differing from any amino acid sequence of the same length of the constitutive Hsc70 amino acid sequence by at least one amino acid located from the fourth to the last C-terminal amino acid of the peptide.

The amino acids are conventionally numbered from the N-terminal extremity of the peptide, corresponding to the free amino function of the first amino acid, to the C-terminal extremity of the peptide, corresponding to the free acid function of the last C-terminal amino acid.

In a more particular embodiment, a peptide according to the invention comprises nine amino acids and its amino acid sequence differs from any amino acid sequence of the same length of the constitutive Hsc70 protein amino acid sequence by at least one amino acid located from the fourth to the eighth position of the peptide.

In another particular embodiment, a peptide according to the invention comprises ten amino acids and its amino acid sequence differs from any amino acid sequence of the same length of the constitutive Hsc70 protein amino acid sequence by at least one amino acid located from the fourth to the ninth position of the peptide.

According to a particular embodiment, a peptide according to the invention comprises an amino acid sequence differing from any amino acid sequence of the same length of the inducible Hsp70 amino acid sequence by at least one amino acid located at position 1, 2 or last C-terminal of the peptide.

In a more particular embodiment, a peptide according to the invention is characterized in that its amino acid sequence from the third amino acid to the one before the last C-terminal amino acid is identical to a portion of the same length within the amino acid sequence of inducible Hsp70.

These differences may be located, within said amino acid sequence, at contiguous or separated positions.

The sequence of the peptide may be modified to artificially increase its immunogenicity. Amino-acid substitutions within the peptides are performed to augment their affinity for HLA class I molecules and facilitate the correct processing of each individual peptide. For instance, the affinity of each peptide is increased by identifying non-favorable amino acids and by their replacement with an alanin shown to result in more potent epitope. Said mutation(s) are intended to increase the immunogenicity of a peptide without affecting its epitopic specificity: said mutations are designed so as to raise the affinity between the epitopic peptide and some HLA molecules but they do not dramatically modify the recognition of the epitope by cytotoxic lymphocytes.

In particular, as the region of an epitopic peptide which mainly interacts with HLA molecules/T cell receptors is located from the third to the C-terminal amino acid of the amino acid sequence of the peptide, mutations located only at positions 1, 2 and/or C-terminal of the peptide, while preserving an identical amino acid sequence from the third to the one before the last C-terminal amino acid, are intended to raise the immunogenicity of the peptide while maintaining the capacity of recognition of the peptide by the cytotoxic T lymphocytes.

Also, sequence additions, particularly on the N-terminal end of the peptides, increases their natural degradation by the proteasome or their transport into the endoplasmic reticulum by the TAP molecules.

In a more particular embodiment of the invention, a peptide as defined above comprises or consists of any amino acid sequence having at least 65% homology with, or identical to, any member of the group of amino acid sequences consisting of:

QVALNPQNTV, LIGRKFGDPV, DMKHWPFQVI, DMKHWPFQV,
VINDGDKPKV, EISSMVLTKM, YLGYPVTNAV, PVTNAVITV,
GVIAGLNVL, VIAGLNVLRI, SILTIDDGI, TIDDGIFEV,
DISQNKRAV, TLSSSTQASL, EIDSLFEGI, DLFRSTLEPV,
TLEPVEKAL, KLDKAQIHDL, LVLVGGSTRI, RIPKVQKLL,
ILMGDKSENV, LMGDKSENV, DLLLLDVAPL, LLLLDVAPL,
LLLDVAPLSL, LLDVAPLSL, DVAPLSLGL, SLGLETAGGV,
GLETAGGVM, ALIKRNSTI, TIPTKQTQI, ALESYAFNM,
LLGRFELSGI, RLSKEEIERM, KISEADKKKV, KVLDKCQEV,
KVLDKCQEVI, VLDKCQEVI, VISWLDANTL, ELEQVCNPI,
ELEQVCNPII, QVCNPIISGL.

In another particular embodiment of the invention, a peptide as defined above comprises or consists of any amino acid sequence having at least 65% homology with, or identical to, any member of the group of amino acid sequences consisting of:

QVALNPQNTV, LIGRKFGDPV, DMKHQPFQVI, DMKHWPFQV,
VINDGDKPKV, YLGYPVTNAV, PVTNAVITV, SILTIDDGI,
DISQNKRAV, TLSSSTQASL, EIDSLFEGI, DLFRSTLEPV,
KLDKAQIHDL, RIPKVQKLL, DLLLLDVAPL, LLLLDVAPL,
LLLDVAPLSL, LLDVAPLSL, DVAPLSLGL, SLGLETAGGV,
ALIKRNSTI, TIPTKQTQI, LLGRFELSGI, RLSKEEIERM,
KISEADKKKV, KVLDKCQEV, KVLDKCQEVI, VLDKCQEVI,
VISWLDANTL, ELEQVCNPI, ELEQVCNPII, QVCNPIISGL.

In another particular embodiment of the invention, a peptide as defined above comprises or consists of any amino acid sequence having at least 65% homology with, or identical to, any member of the group of amino acid sequences consisting of:

ILNVTATDK, SLFEGIDFY, ALNPQNTVF, ILTIDDGIF,
QVSYKGETK, AVITVPAYF, LVNHFVEEF, KVQKLLQDF,
ALESYAFNMK, WLDANTLAEK, RLVNHFVEEF, QVINDGDKPK,
LVNHFVEEFK, FVEEFKRKHK, EVQRERVSAK, AVEDEGLKGK,
KVQKLLQDFF.

In another particular embodiment of the invention, a peptide as defined above comprises or consists of any amino acid sequence having at least 65% homology with, or identical to, any member of the group of amino acid sequences consisting of:

NPQNTVFDA, NPQNTVFDAK, DPVVQSDMK, DPVVQSDMKH,
WPFQVINDG, WPFQVINDGD, KPKVQVSYK, KPKVQVSYKG,
YPEEISSMV, YPEEISSMVL, YPVTNAVIT, YPVTNAVITV,
EPVEKALRD, EPVEKALRDA, IPKVQKLLQ, IPKVQKLLQD,
APLSLGLET, APLSLGLETA, IPTKQTQIF, IPTKQTQIFT,
NPIISGLYQ, NPIISGLYQG, GPGPGGFGA, GPGPGGFGAQ,
GPGGFGAQG, GPGGFGAQGP, GPKGGSGSG, GPKGGSGSGP.

Among Hsp70 derived peptides such as above defined, all possess the necessary primary anchor motifs to bind to HLA-A*0201, HLA-A*0301, or HLA-A*0701 molecules. HLA-A*0201, HLA-A*0301 and HLA-A*0702 being defined as the most represented HLA molecules among Caucasian population. The presence of such motif is necessary to confer a high affinity for such HLA molecules, which is correlated with the immunogenic character of the peptides.

Peptides having, the following amino acids sequence: YLGYPVTNAV (HSP134), LMGDKSENV (HSP380), LLLLDVAPL (HSP391) and LLDVAPLSL (HSP393) are capable of generating a specific cytotoxic T cell immune response in the HLA-A*0201 transgenic murine experimental model (HHD mice).

The peptides HSP134, HSP380, HSP391 and HSP393, restricted by HLA-A*0201, are present at the surface of human tumor cells of various origins over-expressing Hsp70 and are recognized by specific CTL induced in HLA-A*0201 transgenic HHD mice. They also proved to be capable of stimulating specific human CTL which recognize tumors over-expressing Hsp70.

High affinity Hsp70 specific epitopes are also being identified for other HLA molecules, such as HLA-A*0301 or HLA-B*0702.

Preferably, a peptide according to the invention comprises or consists of one of the amino acid sequences: YLGYPVTNAV, LLLLDVAPL and LLDVAPLSL.

The invention also relates to a polyepitope comprising a first amino acid sequence selected from the group of sequences previously defined, and at least a second amino acid sequence that can be:
An amino acid sequence identical to said first amino acid sequence
An amino acid sequence selected from the group of sequences previously cited and that is different from said first amino acid sequence
An epitopic amino acid sequence different from any amino acid sequence selected from the group of sequences previously cited.

A polyepitope is defined as a polypeptide comprising, or consisting of, several epitopes, which are amino acid sequences deriving from antigen processing by the cells, their presentation at the surface of the cells in association with HLA molecules and recognized by lymphocytes. Polyepitopes may include one or several copies of the same epitope, or several different epitopes from the same protein, or epitopes from different antigens. These epitopes may be overlapping, contigous or joined by linkers.

As peptides such as described above, a polyepitope according to the invention may be modified to artificially increase its immunogenicity. Such modifications may be for example amino-acid substitutions or sequence additions, particularly on the N-terminal end.

In a particular embodiment of the invention, a polyepitope has or comprises the amino acid sequence LMGDKSENVQDLLLLDVAPLSL.

Three of the polypeptidic epitopes described above (HSP380, HSP391 and HSP393), selected for their high affinity for HLA-A*0201, form together a sequential 22-mer polyepitope having the amino acid sequence: LMGDKSENVQDLLLLDVAPLSL. These polypeptides and the polyepitope are capable of generating a specific cytotoxic T cell immune response in the HLA-A*0201 transgenic murine experimental model as illustrated in Example 3.

The 22 amino acids natural polyepitope is characterized for its ability to raise 3 distinct immune responses against the three HSP380, HSP391 and HSP393 epitopes in vivo in the HHD murine model, and in vitro from human PBMC (see examples).

In an other embodiment of the invention, a peptide or a polyepitope such as above defined, is linked to a carrier. Said carrier may be a compound associated to a peptide or a polypeptide for its use. For example, a carrier may be intended to raise the immunogenic property of a peptide or a polyepitope, or to facilitate its uptake ex vivo by a phagocyting cell. As an example, this carrier may be a natural or a synthetic biodegradable microparticle. A peptide or a polyepitope according to the invention may possibly be conjugated to the surface of a microparticle.

The invention also relates to a nucleic acid sequence encoding for a peptide or a polyepitope such as above defined.

In a particular embodiment of the invention, this nucleic acid sequence is linked to a vector. This vector may be for example an expression vector adapted to a particular host, or a vector designed to facilitate said nucleic acid uptake by a cell, and particularly an antigen presenting cell. Said vector may be a plasmid, an adenoviral or retroviral vector.

The invention also relates to a method for inducing in vitro a cytotoxic T lymphocyte response that specifically targets cells naturally expressing inducible Hsp70. Said method uses a peptide such as above defined or a polyepitope such as above defined, and may be any method known by a man skilled in the art which is intended to induce in vitro cytotoxic T lymphocytes.

In a particular embodiment of the invention, the method for inducing in vitro this cytotoxic T lymphocyte response uses a nucleic acid encoding for a peptide such as above defined or a polyepitope such as above defined.

The present invention also concerns a cytotoxic T lymphocyte such as obtained by this method. The cytotoxic T lymphocytes according to the invention are able to recognize human tumors through the specific peptides present on human tumor membranes (Example 4).

The present invention also relates to an antigen presenting cell presenting on its surface at least one peptide such as above defined. It also relates to an antigen presenting cells put in contact with a peptide or a polyepitope such as described above.

The peptide or polyepitope may be loaded ex-vivo on human antigen presenting cells (APCs) and effectively activate specific T lymphocytes recognizing the peptides presented by the loaded dendritic cells (Example 5). Such loaded APCs are particularly effective to induce specific cytotoxic T cells in vitro and to induce in vivo a potent antitumoral response mediated by immune effector cells against tumors over-expressing inducible Hsp70. As an example, APCs may be dendritic cells.

Dendritic cells may be prepared by differentiating mononuclear cells, such as purified or partially enriched CD14+ monocytes or PBMCs fractions, anyway obtained by a skilled person from human or animal tissues. For subsequent clinical use, cell collection is carried out by cytapheresis or by density gradient centrifugation of concentrated leukocyte apheresis. Cells are cultivated by standard equipments, flasks and incubators suitable for clinical use.

In a particular embodiment, dendritic cells may be obtained by culture of peripheral blood monocytes and elutriated, according to WO 97/44441, to Boyer et al. (28) or to Bocaccio et al. (29). Briefly, dendritic cells are differentiated in AIMV medium supplemented with 500 U/ml GM-CSF (Leucomax, Novartis Pharma) and 50 ng/ml IL-13 (Sanofi Synthelabo) and elutriated after 7 days of culture. Immature dendritic cells such as obtained may then be “matured” by the use of maturation factors and cytokines known by a man skilled in the art, such as for example IFNγ, poly I:C, ligands of CD40, anti-CD40 antibodies, lipopolysaccharide, agonistic cytokines, including TNFα or FLAT 3 ligand. Immature dendritic cells may also triggered to maturation by using bacterial membrane extracts and/or ribosomal extracts, possibly in association with IFN-γ, using a process described in WO 02/56675.

In a particular embodiment, immature dendritic cells are cultured in complete AIMV medium (Life Technologies, Paisley PA49RF,GB) for 6 hours in presence of Ribomunyl (1 ug/ml) and IFNγ (500 U/ml). Each vial of lyophilised Ribomunyl^{R} (Inava Laboratory, Pierre Fabre, Paris, France) contains 0,010 mg of ribosomal fractions from K. pneumoniae, S. pneumoniae, S. pyogenes and H. influenzae, and 0,015 mg of membrane fractions from K. pneumoniae. IFNγ (Imukin) is obtained from Boehringer Ingelheim France. Cells are washed at 6 hours and further cultured in complete AIMV medium (in absence of maturation factors) until the 40 hours time point. In an other embodiment, immature dendritic cells are incubated in complete AIM medium for 6 hours in the presence of antibody anti-CD40 (2 μg/ml) and poly I:C (100 μg/ml), then washed and further cultured for 34 h.

Dendritic cells may also be obtained by using IFN-α for the differentiation of mononuclear cells. Total PBMCs, partially enriched or highly purified monocytes are then directly cultivated in the presence of type I IFN. Monocytes can be purified by depleting contaminating lymphoid cells using positive immunoselection by anti-CD14 microbeads (MACS Cell Isolation Kits, Miltenyi Biotec, Germany). Alternatively, microbeads conjugated to a monoclonal anti-hapten antibody directed to a cocktail of hapten-conjugated CD3, CD7, CD19, CD45RA and CD56 antibodies (MACS Cell Isolation Kits, Miltenyi Biotec, Germany) are used, as recommended by manufacturer.

In a particularly appropriated procedure, cells are processed and cultured in “closed processors” such as Vaccell® processor (Immuno-Designed Molecules, Paris, France), which include cell cultivation at 37° C. in 5% CO₂ humified air in gas-permeable hydrophobic bags, with medium and autologous serum in the presence of 1,000 IU/ml of type I IFN and 500 U/ml of GM-CSF. rum-free media, human AB or autologous serum can be conveniently used. Different types of standard media (e.g. RPMI-1630, MEM, Iscove's modified Dulbecco's Medium, Dulbecco's modified Eagle Medium) are used according to the subsequent use of DCs, whereas media suitable for treatment of human patients, such as X-VIVO 20 or AIM-V, are preferably used for culturing DCs to be employed in clinical protocols.

Any type I IFN preparation can be used in the generation of IFN-DCs: recombinant IFNα IFNα2b, IFNα2a, natural IFNα(IFNαn) from stimulated leukocytes from healthy subjects or natural lymphoblastoid IFNα, consensus IFN α (CIFN), and recombinant IFNβ. Relevant concentration shall be greater than 100 IU/ml even if ranges of 500-2,000 IU/ml, 500-1,000 IU/ml and particularly a concentration of 1,000 IU/ml are the most preferred. With regard to the up-regulation of costimulatory molecules, the optimal enhancing effects is observed with IFN doses ranging from 500 to 1,000 IU/ml, while 100 IU/ml of IFN does not result in any significant effect. Comparable enhancing effects on DC phenotype are obtained using different preparations of type I IFN such as natural IFN-α, IFNα2b, CIFN and IFNβ, which are added in conjunction with GM-CSF to blood-derived monocytes for 3 days of culture.

Addition of IFN to the culture can be replaced by treatment with any substance capable of inducing type I IFN in culture, provided that the final concentration falls within the ranges above indicated. Timing of the treatment is generally maintained within three days, at the end of which, non-adherent and loosely adherent DCs are collected. Preferably, the cells recovered between day, 2 and day 3 are used directly or purified by either elutriation in a counter current centrifuge or by immunomagnetic negative selection using beads conjugated to lineage specific antibodies. Alternatively, DCs can be conveniently cryopreserved for successive use.

The process may include, following the derivation of DCs from mononuclear cells or from monocytes, a step of further maturation of the DCs, the maturation agent used being chosen among known maturation agents, such as described above.

Dendritic cells may also be obtained from other dendritic cells precursors and by methods known by a man skilled in the art, such as described by Keogh et al. (30).

Antigen presenting cells may be antigen loaded by any method known by a man skilled in the art. In a particular embodiment, dendritic cells may be pulsed for 2 hours with a peptide according to the invention (10 μg/ml) at 37° C. Cells may be pulsed with peptide before the end of maturation time.

A peptide or a polyepitope such as defined above may be modified for better presentation and processing by the antigen presenting cells after injection. For example, peptide or polyepitope may be associated to a particular vector or added with charged amino-acids to the N-terminal or to their C-terminal end.

Nucleic acid as defined before may also be linked to particular vectors such as to be properly loaded on APCs.

The fact that human tumors can be targeted by immunotherapy with the polypeptides of the invention was demonstrated; particularly using different human tumor cell lines shown to over-express inducible Hsp70 and particularly a sufficient expression of the peptides on the membrane.

The Hsp70 epitopes identified can thus advantageously be used for broad-spectrum immunotherapy of cancer, potentially in association with established or under development treatments of cancer, such as surgery, radiotherapy, chemotherapy agents or anti-angiogenic compounds.

The present invention also relates to a pharmaceutical composition or to a vaccine, comprising at least, as active substance, in association with a pharmaceutically acceptable vehicle, a peptide or a polyepitope such as defined above. Said peptide or polyepitope being possibly linked to a vector such as described above.

In a particular embodiment, a pharmaceutical composition or a vaccine according to the invention comprises at least, in association with a pharmaceutically acceptable vehicle, a peptide comprising or consisting of any amino acid sequence selected from the group consisting of: SLFEGIDFY, SLFEGIDFYT, LMGDKSENV.

More particularly, the present invention relates to a pharmaceutical composition or a vaccine according to the invention comprises at least, in association with a pharmaceutically acceptable vehicle, a peptide comprising or consisting the amino acid sequence LMGDKSENV.

Each dose of pharmaceutical composition or vaccine may contain peptide or polypeptide such as defined above, and possibly some other peptides or polyepitopes derived from any antigen known by a man skilled in the art. As an example, a pharmaceutical composition according to the invention may contain an efficient dose of peptide or polyepitope such as defined above and an efficient dose of a peptidic epitope derived from an antigen specifically expressed by some tumor cells. An efficient dose of peptide or polyepitope is comprised between 100 μg and 10 mg of peptide or polyepitope.

In an other embodiment of the invention, the pharmaceutical composition or the vaccine comprises a nucleic acid encoding for said peptide or polyepitope, said nucleic acid being possibly linked to a vector.

Each dose of pharmaceutical composition or vaccine may contain from 100 μg to 10 mg of nucleic acid. Said pharmaceutical composition or vaccine may include an efficient dose of nucleic acid as defined above and an efficient dose of a nucleic acid encoding for any antigen known by a man skilled in the art.

In an other embodiment of the invention, the pharmaceutical composition or the vaccine comprises a cytotoxic T lymphocyte such as obtained by the previously described method.

In an other embodiment of the invention, the pharmaceutical composition or the vaccine comprises an antigen presenting cell presenting on its surface at least one peptide as defined above.

Pharmaceutical composition or vaccine may be administered to a patient under various galenic forms such as intradermal, subcutaneous, intraveinous, intralymphatic, intranodal, intramucosal or intramuscular administration.

The present invention also relates to the use of a pharmaceutical composition or a vaccine according to the invention for the preparation of a drug useful in the treatment of cancer or of a viral disease.

Pharmaceutical compositions and vaccines according to the invention may be used in conjunction with chemotherapy (for example by treatment with cisplatin or 5 FluororUracil) or with anti-angiogenic treatment.

The present invention also relates to the use of any such as described above peptide, polyepitope, nucleic acid, cytotoxic T lymphocyte or antigen presenting cell for the preparation of a drug useful in the treatment of cancer or of a viral disease.

In a particular embodiment, the present invention relates to the use of a peptide comprising or consisting of any amino acid sequence selected from the group consisting of: SLFEGIDFY, SLFEGIDFYT, LMGDKSENV for the preparation of a drug useful in the treatment of cancer or of a viral disease.

### DESCRIPTION OF THE FIGURES AND TABLES

FIG. 1. Identification of the regions comprising the first amino-acid of 9- and 10-mer peptides specifically present in Hsp70 and not present in Hsc70 (□boxed amino-acids).

FIG. 2. Murine CTL avidity for their specific Hsp70 peptide and recognition of their Hsc70 cognate peptides.

The four graphs indicate the cell lysis by CTL lines obtained after stimulation with the following peptides, respectively: HSP134 (line mCTL134), HSP380 (line mCTL380), HSP391 (line mCTL391) and HSP393 (line mCTL393). The avidity of the CTLs for their cellular target is determined with a ⁵¹Cr release assay with an effector:target ratio of 40:1. The percentage of lysis of target cells is expressed on the Y axis, as a function of peptide concentration in the medium, which is represented on the X axis. In each graph, the black curve with diamond-shaped points represent cell lysis in presence of HSP peptides, whereas the grey curve with square points represent cell lysis in presence of cognate HSC peptides.

For measuring the avidity of mCTL134 for cells presenting HSP134, the peptide concentration ranges from 0,01 to 1 mM. For mCTL380, mCTL391 and mCTL393, the respective peptide concentration ranges from 1 pM to 1 μM. The different points on each curve correspond, from left to right, to peptide concentrations of 1 pM, 10 pM, 100 pM, 1 nM, 10 nM, 100 nM and 1 μM.

FIG. 3. Processing of epitopes HSP380, HSP391, HSP393 and HSP134.

After contact of murine CTL lines with transfected COS cell, CTL recognition of COS cells is expressed as CTL secretion of TNF-α dosed in supernatant (X Axis, TNF-α ranges from 0 to 500 pg/ml). The graphs represent TNF-α dosage by cytotoxic cell lines, respectively mCTL380, mCTL391 mCTL393 and mCTL134, after contact with COS cells. In each graph, upper to lower bars represent the different cells in contact with CTL, respectively: negative control (no COS cell), COS cells, COS cells transfected with Hsp70 expression plasmid, COS cells transfected with HHD expression plasmid, positive control (COS cells transfected with HHD and pulsed with the respective peptide), COS cells transfected with HHD and with Hsp70 expression plasmids.

FIG. 4. Screening of tumor cell lines of various origins for their Hsp70 expression. Intracellular inducible Hsp70 expression was assayed by Western Blot, using Actin expression as a relative quantification control. Inducible Hsp70 expression is detected in the upper part of the test, control actin expression is detected in the lower part of the test. The tumor cell lines extracts disposed from left to right are the following:M44, M113, SEG, Caco-2, SAOS, MCF-7.

FIG. 5. Recognition by murine CTL of the epitopes at the surface of human tumor cells over-expressing Hsp70.

CTL recognition of tumoral cells is expressed as CTL secretion of TNF-α dosed in supernatant (X Axis, TNF-α ranges from 0 to 1000 pg/ml) after contact with the cells. Upper to lower bars represent the different cells and reagents in contact with CTL, respectively: 2 negative controls (M113, M44), SAOS, MCF-7, MCF-7 blocked with anti-class I (w6/32) antibody before mixed culture, and MCF-7 blocked with an irrelevant antibody before mixed culture.

FIG. 6. Induction of specific human CTL by epitopes HSP391 and HSP393.

Specificity was verified in a ⁵¹Cr release assay on target cells pulsed with 101M of peptide. The X axis represents the CTL/target cell ratio, namely 20/1 and 10/1. The Y axis represents the percentage of target cell lysis. The upper and lower graph represents results obtained respectively with human CTL hCTL391 and hCTL393. The black curves with diamond-shaped points correspond to cell lysis in presence of the relevant peptide, namely HSP391 or HSP393. The grey curve with square points correspond to cell lysis in presence of an irrelevant peptide (HIV Pol peptide).

FIG. 7A. Capacity of recognition of cells loaded with Hsp70 peptides by human CTL specific for the corresponding peptides,

The three graphs entitled hCTL380, hCTL391 and hCTL393 represent respectively the testing of p380, p391 and p393-specific human CTLs. P391 and p393 specific CTLs were tested towards EBV-B cells, whereas p380 specific CTLs were tested towards T2 cells. The number of IFN-γ producing CD8+ cells for 10⁵ CD8+ cells upon activation, indicated on the X-axis, was measured, as assessed by staining cells for CD8 and intracellular IFN-γ.

EBV-B or T2 cells were loaded either with p380, p391, p393. EBV-B or T2 cells loaded with irrelevant HrVpol₅₈₉ peptide were used as negative controls.

FIG. 7B. Recognition of Hsp70 expressing human tumor cells by specific human CTL.

The two graphs, entitled hCTL391 and hCTL393, represent respectively the testing of p391 and p393-specific human CTLs. P391 and p393 specific CTLs were tested for their recognition of the Hsp70 expressing human tumor cells MCF-7 and SAOS by measuring IFN-γ producing CD8+ cells upon activation, as assessed by staining cells for CD8 and intracellular IFN-γ. The number of IFN-γ producing CD8+ cells for 10⁵ CD8+ cells upon activation It is indicated on the X-axis.

EBV-B or T2 cells were loaded either with p380, p391, p393.

Hsp70 negative M44 and M113 tumor cells were used as negative controls.

FIG. 8A. Immunogenicity of a polyepitope: Induction of multiple immune responses against epitopes p380, p391 and p393 upon vaccination of HHD mice.

The left graph shows results obtained after immunization of HHD mice with the polyepitope, whereas the right graph shows results obtained following immunization of HHD mice with a mix of peptides p380, p391 and p393. Immune responses were monitored ex vivo in an IFN-γ Elispot assay. The X axis represents the percentage of immunized mice responding to the different epitopes. On the Y axis, upper to lower bars represent the total percentage of mice exhibiting an immune response against any epitope, then the percentage of mice responding, respectively, against p380, p391 and p393 peptides.

FIG. 8B. Frequency of specific IFN-γ secreting cells in responding mice.

The 8 graphs represent the immune responses obtained for each of the immunized mice with the 22-mer epitope. The X axis represents the number of IFN-γ producing CTLs for 10⁵ ex vivo CD8+ cells upon activation. On the Y axis, the bars represent the respective level of IFN-γ producing CTLs against p391 or p393 peptide or against irrelevant HIV pol589 peptides.

FIG. 9. Immunogenicity of polyepitope in a human setting through induction of immune responses in vitro against p380, p391 or p393.

Polyepitope induced human CTL were tested for their capacity to respond to T2 cells loaded with p380, p391 or p393 or the irrelevant HIVpol589 peptides in intracellular IFN-γ staining. The X axis represents the number of IFNγ-producing CTL/10⁵ CD8+ cells. On the Y axis, upper to lower bars represent the results obtained when cells were loaded respectively with irrelevant HIV pol589 peptides, or with p380, p391 or p393 peptides.

### EXAMPLES

Materials and Methods

Measurement of Peptide Relative Affinity to HLA class I. Tap−/− cells expressing the studied HLA-class I molecule (3×10⁵ cells/mL) were incubated with various concentrations of peptides ranging from 100 μM to 0.1 μM in serum-free RPMI 1640 medium supplemented with 100 ng/ml of human β-2m at 37° C. for 16 hours. Cells were then washed twice and stained with the specific anti-class I mAb followed by FITC conjugated goat anti mouse Ig mAb to quantify the expression of HLA-A*0201. For each peptide concentration, the HLA-specific staining was calculated as the % of the staining obtained with 100 μM of the reference peptide. The relative affinity (RA) is determined as: RA=(Concentration of each peptide that induces 20% of HLA expression/Concentration of the reference peptide that induces 20% of HLA expression). The lower the RA-value, the stronger is the peptide binding to HLA. The mean RA value for each peptide is determined from at least three independent experiments.

For the study of Relative Affinity to HLA-A*0201, Tap−/− HLA-A*0201 T2 cells, the HLA-A*0201 specific mAb BB7.2 and the reference HLA-A*0201-binding peptide HIVpol 589 (IVGAETFYV) were used. For the study of Relative Affinity to HLA-A*0301, Tap−/− HLA-A*0301 T2-A3 cells are used. For the study of Relative Affinity to HLA-B*0701, Tap−/− HLA-B*0701 T2-B7 cells are used.

Assessment of Peptide/HLA class I Complex Stability. Tap−/− cells (10⁶/mL) were incubated overnight with 100 μM of each peptide in serum-free RPMI 1640 medium supplemented with 100 ng/mL of P-2m at 37° C. Cells were then washed four times to remove free peptide, incubated with Brefeldin A (10 μg/mL) for 1 hour to block cell surface expression of newly synthesized HLA molecules, washed and incubated at 37° C. for 0, 2, 4 or 6 hours. Subsequently, cells were stained with the HLA specific mAb followed by FITC conjugated goat anti mouse Ig mAb. For each time point, peptide induced HLA expression was calculated as: Mean fluorescence of peptide preincubated cells—Mean fluorescence of cells treated in similar conditions in the absence of peptide. DC50 (dissociation complex; DC) was defined as the time required for the loss of 50% of the HLA-A*0201/peptide complexes stabilized at t=0.

Generation of CTL in HLA-transgenic mice. HLA-transgenic mice were injected subcutaneously at the base of the tail with 100 μg of peptide emulsified in incomplete Freund's adjuvant (IFA) in the presence of 140 μg of the I-A^{b} restricted HBVcore-derived T-helper epitope (128-140; sequence TPPAYRPPNAPIL). After 11 days, spleen cells (5×10⁷ cells in 10 mL) were stimulated in vitro with peptide (10 μM). On day 6 of culture, the bulk responder populations were tested for specific cytotoxicity. Upon response, the CTL line was then weekly re-stimulated in vitro with 20×10⁶ 35Gy irradiated spleen cells in the presence of 1 to 0.1 μM peptide and 50 U/mL IL-2 (Proleukin, Chiron Corp.). Cytotoxicity was assayed 6 days after the last stimulation and TNF-α secretion 11-13 days after, with the addition of 50 U/mL IL-2 on day 7. HHD mice transgenic for HLA-A*0201 were used. B7B7 Kd mice and mice transgenic for HLA-A*0301 are used for the study of the HLA-B*0701 and HLA-A*0301 restricted peptides respectively.

Generation of CTL from Human Peripheral Blood Mononuclear Cells (PBMC). Fresh PBMC collected from healthy donnors under informed consent were serologically typed for the studied HLA-class I molecule, and purified using standard Ficoll-Paque (Amersham Pharmacia Biotech AB). Dendritic cells (DC) were prepared by cultivating plastic adherent PBMC in the presence of 500 U/mL GM-CSF (R&D Systems Inc. MN) and 500 U/mL IL-4 (R&D Systems Inc. MN) for 7 days such as described in Keogh et al. (30).

DC were then maturated for 2 days with 2 μg/mL anti-CD40 antibody and 100 ng/mL Poly I:C (Sigma-Aldrich). Immunofluorescence staining of this DC-enriched population showed that >90% were CD86/B7-2+ and HLA-DR+cells. Autologous CD8+ purified T-cells (CD8 MicroBeads, Miltneyi Biotec Inc. CA) were stimulated weekly with DC pulsed for two hours with peptide (10 μM) and irradiated at 35Gy at a T:DC ratio of 10:1 in culture medium (CM; RPMI 1640 supplemented with 10% human AB serum, 10 nM L-glutamine and gentamycin). 1×10³ U/mL IL-6 and SU/mL IL-12 (R&D Systems Inc. MN) were added during the first week of culture. 20 U/mL clinical grade IL-2 (Proleukin, Chiron Corp.) and 10 ng/mL IL-7 (R&D Systems Inc. MN) were added during the next two weeks of culture. 7 days after the last stimulation with DC, peptide specific IFN-γ secreting CD8+ T cells were magnetically sorted following encounter of the peptide presented by HLA-A*0201+ B-EBV cells (IFN-γ Secretion Assay, Miltenyi Biotec Inc. CA). The characterization of peptide-specific T cells was tested 7 days after the purification step.

Identification of Hsp70-specific TILs from cancer patients. Fresh PBMC collected from cancer patients under informed consent were serologically typed for the studied HLA-class I molecule, and purified using standard Ficoll-Paque (Amersham Pharmacia Biotech AB). Tumor infiltrating lymphocytes and tumor cell lines were prepared by dissociation of solid tumor minced small fragments with collagenase solution. Bulk tumor lines were cultured in 10% FCS DMEM culture medium, and eventually cloned. Furthermore, T lymphocytes were obtained from freshly resected tumor-infiltrated lymph nodes, crushed to release cells into culture.

The detection of Hsp70 specific TILs was first addressed through detection of TNF-α secretion upon encounter of COS cells transfected with both HLA class I molecules and Hsp70 expression plasmids and of Hsp70-peptide tetramer positive CD8+ cells.”.

The identification of TILs specific for known Hsp70 peptides was addressed with the ELISPOT assay. To extend the sensitivity of this assay, lymphocytes were stimulated once in vitro prior to analysis (22). At day 0, PBLs or crushed lymph nodes were thawed and plated in 2 mL/well at a concentration of 2×10⁶ cells in 24-well plates (Nunc) in 10% human AB serum culture medium in the presence of 10 mM of peptide. In each experiment, a well without peptide was also included. Two days later, 300 IU/mL clinical grade interleukin 2 (Proleukin, Chiron Corp.) were added to the cultures. The cultured cells were tested for reactivity in the ELISPOT on day 12.

ELISPOT Assay. The ELISPOT assay was used to quantify peptide epitope-specific, IFN-γ-releasing effector cells. Mixed cellulose ester membrane 96-well plates (MultiScreen MAHA S4510; Millipore) were coated over-night with anti-IFN-γ antibody B285). The wells were washed and blocked by 10% human AB serum culture medium, and cells were added in quadmplicates at different cell concentrations. Peptides were then added to each well, and the plates were incubated over-night. The following day, media were discarded, and the wells were washed prior to addition of biotinylated secondary antibody (BAF285-Biotin). The plates were incubated for 2 h and washed, and streptavidin-enzyme conjugate (Streptavidin-AP; Boehringer Mannheim GmbH) was added to each well. Plates were incubated at room temperature for 1 h, and the enzyme substrate BCIP-NBT (S3771; Promega, France) in pH 9.5 phosphatase alcaline buffer (100 mM tris HCl, 100 mM NaCl, 5 mM MgCl₂) was added to each well and incubated at room temperature for 10-20 min. The reaction was terminated by washing with tap water upon the emergence of dark purple spots. The spots were counted using an automated image analysis system ELISPOT Reader (A/D Strassberg, Germany). The peptide-specific IFN-γ secreting cells frequency could be calculated from the numbers of spot-forming cells and from the CD8+ frequency in the studied lymphocyte population, adressed by immunostaining with an anti-CD8 mAb (clone 3B5; Caltag Laboratories, CA).

The assays were all performed in quadruplicates for each peptide antigen.

Cytotoxic assay. Targets were labeled with 100 μCi of ⁵¹Cr for 90 min, washed 3 times and plated in 96-well V-bottomed plates (2.5×10³ cells/well in 100 μL of RPMI 1640+5% FCS). They were pulsed with peptides at 37° C. for 60 min. 100 L of various numbers of effectors were then added in the wells and incubated at 37° C. for 4 hours. After incubation, 100 μL of supernatant was collected and radioactivity was measured in a γ-counter. Percentage of specific lysis was determined as: Lysis=(Experimental Release−Spontaneous Release)/(Maximal Release−Spontaneous Release)×100. Spontaneous release was always <20% of maximal release induced by 3N HCl.

TAP deficient murine RMAS-HHD and human T2 cells were used as HLA-A*0201 targets for cytotoxicity. Murine P815-B7 and human T2-B7 cells were used as HLA-B*0701 targets for cytotoxicity.

Peptide Processing Assay on COS-Transfected Cells. 2.2×10⁴ simian COS cells were plated in flat-bottom 96 well plates in DMEM 10% FCS, in quadruplicates for each condition. 18 hours later, the medium is discarded, and 100 ng of each DNA plasmid, encoding for Hsp70 and HLA class I molecules, is put in contact with COS cells in DMEM 10% Nuserum 10 mM Chloroquine 10 mg/mL DEAE Dextran. After 4 hours of incubation at 37° C., transfection medium is discarded, and 50 μL PBS 10% DMSO is added for 2 minutes for integration of plasmids. It is then discarded and transfected COS cells are placed in DMEM 10% FCS for 40 hours. Transfected COS cells are then used with 5×10⁴ murine CTL in a TNF-α secretion assay.

TNF-α Secretion Assay. Transfected COS cells on day 4 or 10⁵ target tumor cells were plated in 50 μL RPMI 10% FCS and when necessary incubated with 10 μM peptide for 1 hour. In blocking experiments, anti-HLA class I antibody w6/32 or an irrelevant antibody were incubated with target cells for 1 h30. 5×10⁴ T cells were then added in 50 μL RPMI 10% FCS and incubated for 6 hours. Each condition was tested in quadruplicates. 50 μL of the supernatant was collected. Standard dilutions were prepared in 50 μL with final doses of TNF-α (kindly provided by I. Apfler, Bender Wien, Austria) ranging from 10⁴ to 0.08 pg/mL. On both the supernatants and the standards were added 50 μL of 3×10⁴ TNF-α sensitive WEHI-164c13 cells. They were incubated for 16 hours at 37° C. (23). Inhibition of cell proliferation was evaluated by the MTT colorimetric method. Briefly, 50 μL of MTT at 2.5 mg/mL (Sigma-Aldrich) was added and incubated for 4 hours at 37° C. Cells were then lyzed with 100 μL of a pH4.7 33% N,N-dimethyl formamide (Sigma-Aldrich) 20% SDS solution in water. Two hours after cells lysis, OD was measured at 550 nm on a Dynatech MR5000 spectrophotometer. TNF-α quantitation was calculated by linear interpolation with the standard dilutions.

Western Blot Analysis of Hsp70 Expression by Tumor Cells. Cellular samples were rinsed in PBS, then lysed 30 min at 4° C. in 125 mM Tris/HCl pH 6.8 containing 3 mM EDTA, 10 mM NaF and 0.1% sulfobetain 14. After centrifugation (13000 rpm, 10 min, 4° C.), supernatants were quantified for their protein content using BCA assay (Pierce). 30 μg of proteins were loaded on 5-15% SDS-PAGE gradients under denaturing conditions, and transferred to nitrocellulose membranes. Detection of HSP70, and actin was done by incubating the membranes 1 h at 37° C. with the appropriated amount of antibody (anti HSP70, Stressgen, dilution 1:1000, anti actin, Chemicon, dilution 1:2000). The fixation of primary antibodies was detected using a peroxydase-conjugated anti mouse Ig (Sigma), and the ECL kit (Amersham Pharmacia).

Intracellular IFN-g Staining. 5×10⁴ T cells were incubated with 10⁵ stimulating peptide loaded EBV-B cells or with 10⁵ tumor cells in the presence of 20 μg/ml brefeldin-A (Sigma, Oakville, Canada). Six hours later, they were washed, stained with r-phycoerythrin-conjugated anti-CD8 antibody (Caltag Laboratories, Burlingame, Calif.) in PBS for 25 min at 4° C., washed and fixed with 4% PFA. Then, cells were permeabilized with PBS 0.5% BSA 0.2% saponin (Sigma, Oakville, Canada), and stained with allophycocyanin-conjugated anti-IFNg mAb (PharMingen, Mississauga, Canada) for 25 min at 4° C. Cells were analyzed on FACSCalibur (Becton Dickinson, Mountain View, Calif.).

Results

### Example 1

### Selection of Specific Hsp70 Regions

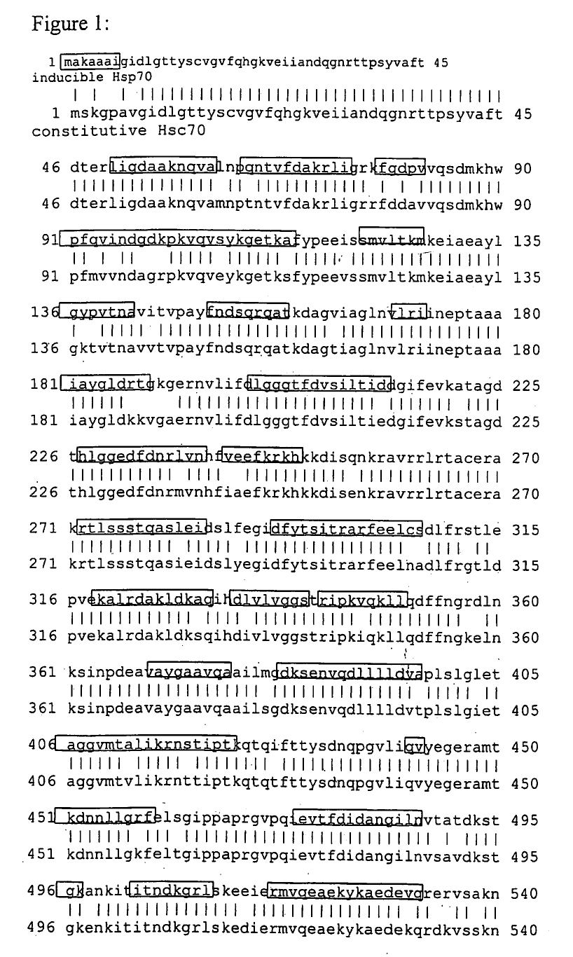

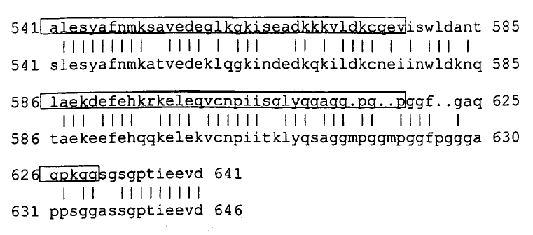

Amino acid sequences of human inducible Hsp70 and constitutive Hsc70 were aligned, and mismatches were set off (FIG. 1). 9- or 10-mer peptides containing at least one mismatch formed regions on which work was focused hereafterwere selected.

Regions comprising amino-acids 1-7, 52-64, 68-81, 85-119, 128-144, 154-163, 177-191, 204-221, 228-243, 246-240, 246-255, 273-288, 297-315, 320-334, 337-346, 348-358, 372-381, 388-427, 449-462, 480498, 505-514, 522-632 define the first amino-acid of all such peptides.

Among the peptides belonging to these Hsp70 specific regions, we selected peptides that possessed the necessary anchor motifs to bind to HLA-A*0201 (L, M, V or I in position 2 and L, V, M or I in C-terminal position), to HLA-A*0301 (L, V or M in position 2 and K, Y or F in C-terminal position), and to HLA-B*0702 (P in position 2).

a) Peptides with HLA-A*0201 Anchor Motifs: 42 Peptides

tlepvekal
lllldvapl
lldvaplsl
gletaggvm
alesyafnm
alikrnsti
vldkcqevi
eleqvcnpi
dmkhwpfqv
lmgdksenv
gviaglnvl
dvaplslgl
pvtnavitv
kvldkcqev
ripkvqkll
tiddgifev
disqnkrav
siltiddgi
eidslfegi
tiptkqtqi
tlssstqasl
kldkaqihdl
dlllldvapl
llldvaplsl
rlskeeierm
ylgypvtnav
dlfrstlepv
ilmgdksenv
slgletaggv
llgrfelsgi
eleqvcnpii
dmkhwpfqvi
qvcnpiisgl
qvalnpqntv
lvlvggstri
kvldkcqevi
viswldantl
eissmvltkm
ligrkfgdpv
vindgdkpkv
kiseadkkkv
viaglnvlri

b) Peptides with HLA-A*0301 Anchor Motifs: 17 Peptides

ilnvtatdk
slfegidfy
alnpqntvf
iltiddgif
qvsykgetk
avitvpayf
lvnhfveef
kvqkllqdf
alesyafnmk
wldantlaek
rlvnhfveef
qvindgdkpk
lvnhfveefk
fveefkrkhk
evqrervsak
avedeglkgk
kvqkllqdff

c) Peptides with HLA-B*0702 Anchor Motifs: 28 Peptides

| | | |
|---|---|---|
| Position number | Hsp | Hsc |

| | | |
|---|---|---|
| 62 | npqntvfda | nptntvfda |
| 62 | npqntvfdak | nptntvfdak |
| 80 | dpvvqsdmk | davvqsdmk |
| 80 | dpvvqsdmkh | davvqsdmkh |
| 90 | wpfqvindg | wpfmvvnda |
| 90 | wpfqvindgd | wpfmvvndag |
| 100 | kpkvqvsyk | rpkvqveyk |
| 100 | kpkvqvsykg | rpkvqveykg |
| 115 | ypeeissmv | ypeevssmv |
| 115 | ypeeissmvl | ypeevssmvl |
| 137 | ypvtnavit | ktvtnavvt |
| 137 | ypvtnavitv | ktvtnavvtv |
| 315 | epvekalrda | dpvekalrda |
| 315 | epvekalrda | dpvekalrda |
| 343 | ipkvqkllq | ipkiqkllq |
| 343 | ipkvqkllqd | ipkiqkllqd |
| 397 | aplslglet | tplslgiet |
| 397 | aplslgleta | tplslgieta |
| 420 | iptkqtqif | iptkqttf |
| 420 | iptkqtqift | iptkqtqtft |
| 604 | npiisglyq | npiitklyq |
| 604 | npiisglyqg | npiitklyqs |
| 616 | gpgpggfga | ggmpggfpg |
| 616 | gpgpggfgaq | ggmgppfpgg |
| 618 | gpggfgaqg | mpggfpggg |
| 618 | gpggfgaqgp | mpggfpggga |
| 626 | gpkggsgsg | ppsggassg |
| 626 | gpkggsgsgp | ppsggassgp |

It is to be noted that the expression “Position number” corresponds to the number of the position of the first amino acid of the peptide in the corresponding polypeptide (Hsp or Hsc).

9- or 10-mer peptides containing at least one mismatch between their forth and C-terminal position formed regions on which work was focused hereafter:

d) Peptides with HLA-A*0201 Anchor motifs and mismatch Hsp70/Hsc70 Between Their Forth and C-Terminal Position: 32 Peptides

| | | |
|---|---|---|
| Position number | Hsp | Hsc |

| | | |
|---|---|---|
| 58 | qvalnpqntv | qvamnptntv |
| 72 | ligrkfgdpv | ligrrfddav |
| 85 | dmkhwpfqvi | dmkhwpfmvv |
| 86 | dmkhwpfqv | dmkhwpfmv |
| 94 | vindgdkpkv | vvndagrpkv |
| 134 | ylgypvtnav | ylgktvtnav |
| 138 | pvtnavitv | tvtnavvtv |
| 207 | siltiddgi | siltiedgi |
| 252 | disqnkrav | disenkrav |
| 273 | tlssstqasl | tlssstqasi |
| 283 | eidslfegi | eidslyegi |
| 307 | dlfrstlepv | dlfrgtldpv |
| 325 | kldkaqihdl | kldksqihdi |
| 342 | ripkvqkll | ripkiqkll |
| 390 | dlllldvapl | dlllldvtpl |
| 391 | lllldvapl | lllldvtpl |
| 392 | llldvaplsl | llldvtplsl |
| 393 | lldvaplsl | lldvtplsl |
| 395 | dvaplslgl | dvtplslgi |
| 400 | slgletaggv | slgietaggv |
| 412 | alikrnsti | vlikrntti |
| 419 | tiptkqtqi | tiptkqtqt |
| 455 | llgrfelsgi | llgkfeltgi |
| 509 | rlskeeierm | rlskedierm |
| 561 | kiseadkkkv | kindedkqki |
| 569 | kvldkcqev | kildkcnei |
| 569 | kvldkcqevi | kildkcneii |
| 570 | vldkcqevi | ildkcneii |
| 576 | viswldantl | iinwldknqt |
| 598 | eleqvcnpi | elekvcnpi |
| 598 | eleqvcnpii | elekvcnpii |
| 601 | qvcnpiisgl | kvcnpiitkl |

e) Peptides with HLA-A*0301 Anchor Motifs and Mismatch Hsp70/Hsc70 Between and C-Terminal Position: 10 Peptides.

| | | |
|---|---|---|
| Position number | Hsp | Hsc |

| | | |
|---|---|---|
| 485 | ilnvtatdk | ilnvsavdk |
| 60 | alnpqntvf | amnptntvf |
| 209 | iltiddgif | iltiedgif |
| 237 | lvnhfveef | mvnhfiaef |
| 580 | wldantlaek | wldkngtaek |
| 236 | rlvnhfveef | rmvnhfiaef |
| 93 | qvindgdkpk | mvvndagrpk |
| 237 | lvnhfveefk | mvnhfiaefk |
| 530 | evqrervsak | ekqrdkvssk |
| 552 | avedeglkgk | tvedeklqgk |

f) Peptides with HLA-B*0702 Anchor Motifs and Mismatch Hsp70/Hsc70 Between and C-Terminal Position: 22 Peptides

| | | |
|---|---|---|
| Position number | Hsp | Hsc |

| | | |
|---|---|---|
| 90 | wpfqvindg | wpfmvvnda |
| 90 | wpfqvindgd | wpfmvvndag |
| 100 | kpkvqvsyk | rpkvqveyk |
| 100 | kpkvqvsykg | rpkvqveykg |
| 115 | ypeeissmv | ypeevssmv |
| 115 | ypeeissmvl | ypeevssmvl |
| 137 | ypvtnavit | ktvtnavvt |
| 137 | ypvtnavitv | ktvtnavvtv |
| 343 | ipkvqkllq | ipkiqkllq |
| 343 | ipkvqkllqd | ipkiqkllqd |
| 397 | aplslglet | tplslgiet |
| 397 | aplslgleta | tplslgieta |
| 420 | iptkqtqif | iptkqtqtf |
| 420 | iptkqtqift | iptkqtqtft |
| 604 | npiisglyq | npiitklyq |
| 604 | npiisglyqg | npiitklyqs |
| 616 | gpgpggfga | ggmpggfpg |
| 616 | gpgpggfgaq | ggmpggfpgg |
| 618 | gpggfgaqg | mpggfpggg |
| 618 | gpggfgaqgp | mpggfpggga |
| 626 | gpkggsgsg | ppsggassg |
| 626 | gpkggsgsgp | ppsggassgp |

### Example 2

### Selection of Specific HSP Sequences with High Affinity

Despite its basal expression profile in normal cells, it cannot be excluded that Hsp70 belongs to the immunological self and that Hsp70 epitopes presented in the thymus or in the periphery have tolerized specific CD8+ repertoires. If peptides also belong to the immunological self of the HLA-A*0201 transgenic HHD mice, such a model enables to study ex vivo tolerization phenomena. Therefore, the approach followed to identify immunogenic Hsp sequences restricted the study to Hsp70 peptides shared with the murine Hsp70 sequence.

The sequence of the human inducible Hsp70 protein was screened for HLA-A*0201 binding peptides with a computerized algorithm. Four 9- or 10-mer peptides, predicted as strong HLA-A*0201 binders, not present in Hsc70 and present in the murine Hsp70, were selected to be studied experimentally for their HLA-A*0201 affinity and stability. Their position in the human Hsp70 protein and sequence is summarized in the following Table 1. The experimental Relative Affinity describes the peptide dose necessary to obtain a reference number of HLA-A*0201/peptide complexes relatively to the same dose of the reference peptide HIV Pol. Stability of the HLA-A*0201/peptide complex is determined as half life of the complex upon removal of the source of exogenous peptide.

| | | | | |
|---|---|---|---|---|
| | | Relative | Stability | Homologous |
| Position | Sequence | affinity | (hrs) | Hsc 70 |

| | | | | |
|---|---|---|---|---|
| HSP 73 | LIGRKFGDPV | >20 | | LIGRRFDDAV |
| HSP 134 | YLGYPVTNA | 2.5 | | YLGKTVTNA |
| HSP | YLGYPVTNAV | 0.6 | | YLGKTVTNAV |
| 134(10) | | | | |
| HSP 286 | SLFEGIDFYT | 0.25 | >6 h | SLYEGIDFYT |
| HSP 380 | LMGDKSENV | 2.5 | 4 h | LSGDKSENV |
| HSP 391 | LLLLDVAPL | 0.5 | >6 h | LLLLDVTPL |
| HSP 393 | LLDVAPLSL | 0.4 | n/d | LLDVTPLSL |

The affinity of each peptide was assayed experimentally by determination of the Relative Affinity: the necessary peptide dose to stabilise a reference quantity of HLA-A*0201 molecules at the surface of T2 cells; and the stability of each peptide was assayed by determination of the half-life of the peptide/HLA-A*0201 molecule complex (DC50: dissociation complex 50).

All four candidates presented the desired profile of a Relative Affinity <5, an affinity high enough to ensure their immunogenicity. It was previously demonstrated that more than 98% of peptides with a relative affinity below 5 are immunogenic (27).

3 candidates presenting this latter profile are all three located within 22 amino-acids. They form a natural 22-mer polyepitope: HSP380, HSP391 and HSP393.

Study of peptides was also extended to peptides which are not present in the sequence of the murine Hsp70: the determination of the Relative Affinity for such peptides lead to the identification of a peptide, HSPI34(10), with a strong affinity for HLA-A*0201 (table 1). This peptide is equally of interest in humans.

High affinity peptide discovery was also lead for the MHC molecule HLA-B*0702. 11 peptides presenting the primary anchor motif for HLA-B*0702 or predicted to show a high affinity by the computerized program BIMAS were tested for their Relative Affinity. One peptide, HSP137, showed a high affinity for HLA-B*0702. The following Table 2 shows the sequence of Hsp70 peptides shared with the murine Hsp70, their cognate Hsc70 peptide and their relative affinity towards HLA-B*0702 molecule.

| | | | |
|---|---|---|---|
| Position | Sequence | Relative affinity | Homologous Hsc 70 |

| | | | |
|---|---|---|---|
| HSP 115 | YPEEISSMV | >10 | YPEEVSSMV |
| HSP 115(10) | YPEEISSMVL | >10 | YPEEVSSMVL |
| HSP 137 | YPVTNAVIT | 1.2 | KTVTNAVVT |
| HSP 137(10) | YPVTNAVITV | >10 | KTVTNAVVTV |
| HSP 340 | STRIPKVQKL | >10 | STRIPKIQKL |
| HSP 395 | DVAPLSLGL | >10 | DVTPLSLGI |
| HSP 397 | APLSLGLET | >10 | TPLSLGIET |
| HSP 420 | IPTKQTQIF | >10 | IPTKQTQTF |
| HSP 420(10) | IPTKQTQIFT | >10 | IPTKQTQTFT |
| HSP 601 | QVCNPIISGL | >10 | KVCNPIITKL |
| HSP 616 | GPGPGGFGA | >10 | GGMPGGFPG |

### Example 3

### Immunogenicity of Inducible HSP70 Polypeptides in HHD Mice

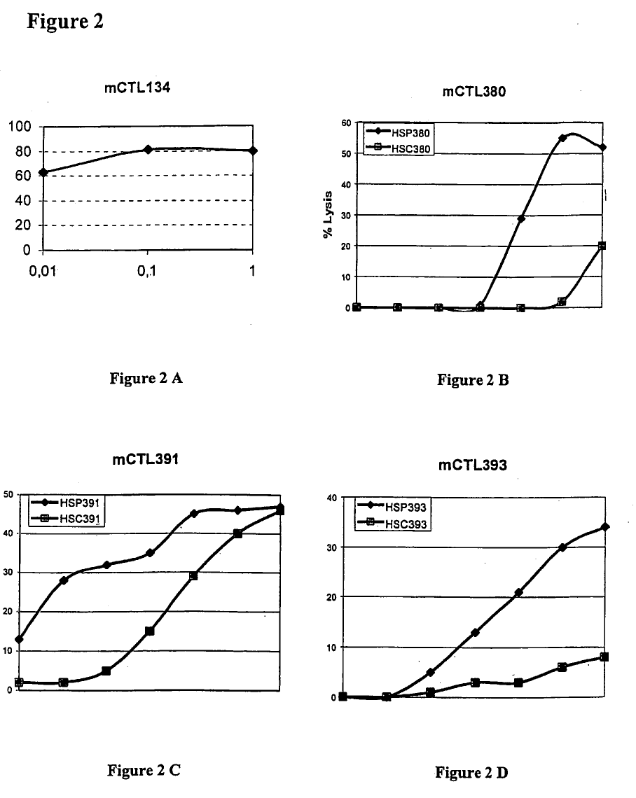

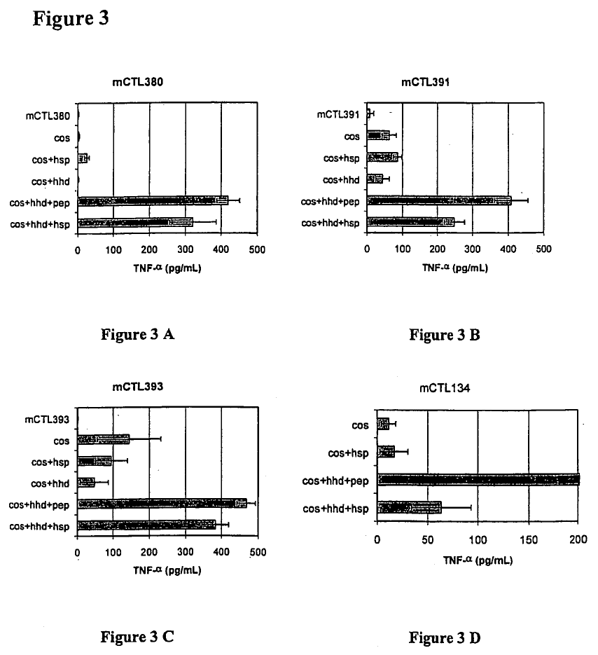

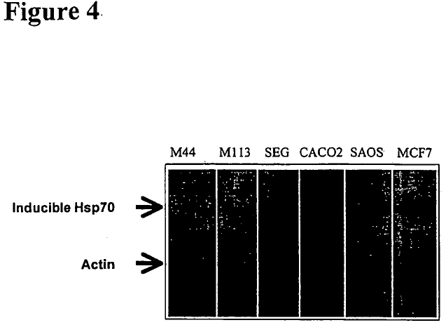

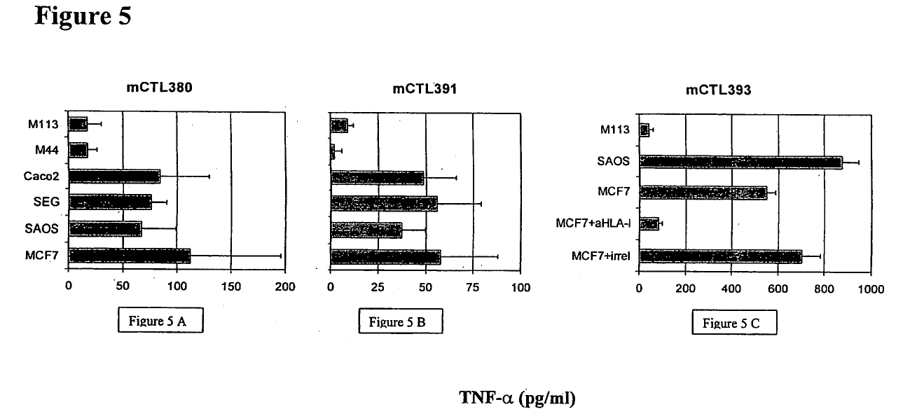

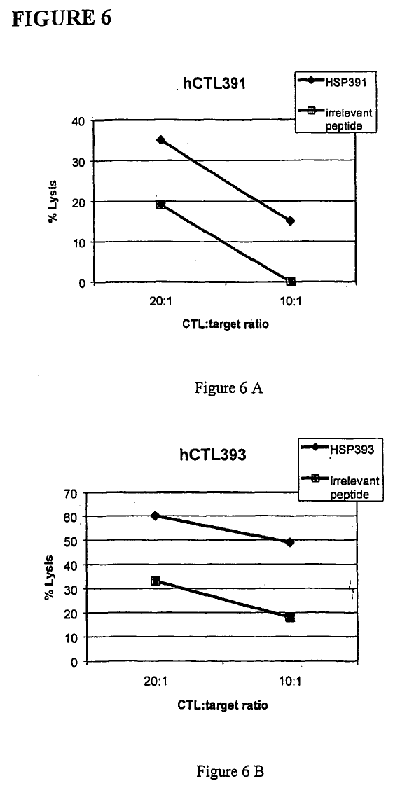

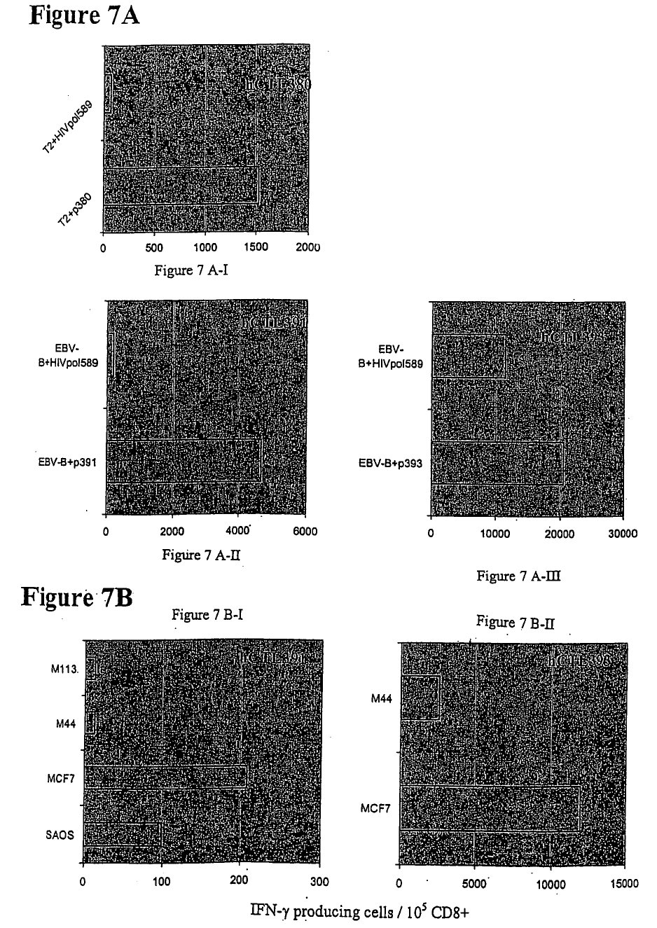

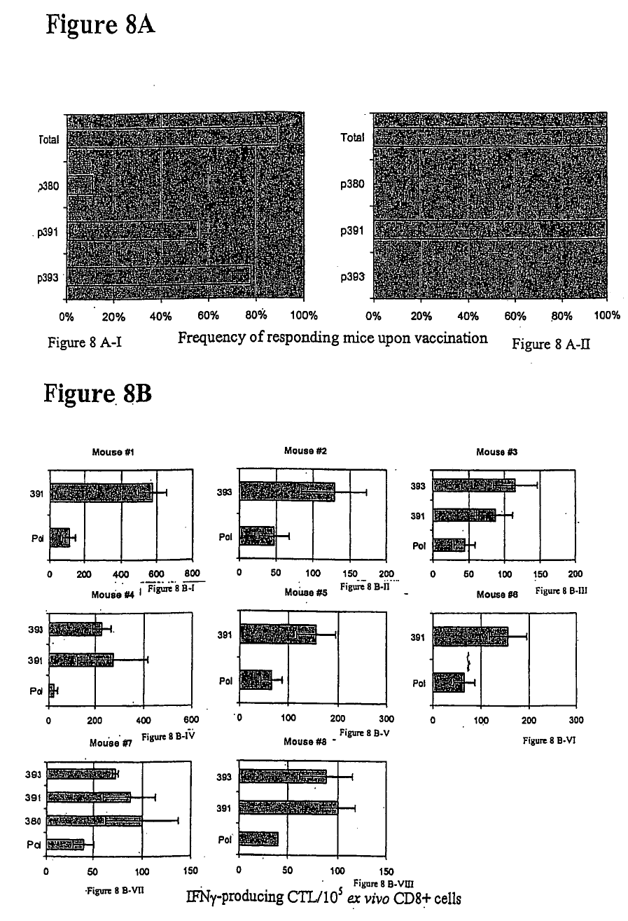

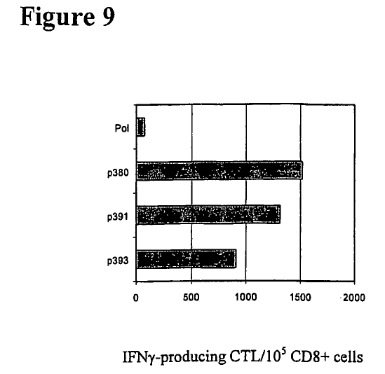

We studied the immunogenicity of the 4 HLA-A*0201 restricted Hsp70 peptides selected in HHD mice. CTLs were induced against all four peptides in vivo and high avidity CTL lines were obtained after several in vitro restimulations. Indeed, each CTL line recognize its respective Hsp70 peptide with an avidity (50% of maximal lysis) of about 10 nM for HSP380, and below 10 nM for HSP134, HSP391 and HSP393. However, they recognized only weakly the Hsc70 cognate peptides, as the avidity for these latter peptides is always about 3 logs higher than for the Hsp70 cognate peptide (FIG. 2). These CTL lines are respectively referred to as mCTL134, mCTL380, mCTL391 and mCTL393.

The four peptides selected were correctly processed and indeed shown to be epitopes. In the COS model which enables a high expression of both Hsp70 and HHD proteins, all four cell lines mCTL134, mCTL380, mCTL391 and mCTL393 clearly and specifically recognized COS cells transfected with both Hsp70 and HHD expression vectors (FIG. 3). We thus clearly demonstrate that all four HSP134, HSP380, HSP391 and HSP393 are epitopes.

### Example 4

### Tumors Over-Expressing Inducible Hsp70 are Recognized by the Cytotoxic Lymphocytes Generated Against the Specific Polypeptides

We studied whether the over-expression of Hsp70 described in certain tumor cell lines was high enough to enable these epitopes to be presented at the surface of tumor cells. We addressed this question with our already available high-avidity murine CTLs, hypothesizing that such a high CTL avidity enables the recognition of the highest-Hsp70 expressing human tumor cells. We screened human tumor cell lines for their HLA-A*0201 and Hsp70 expression and identified a pool of four HLA-A*0201+Hsp70+tumors of various types:
breast cancer (MCF-7), sarcoma (SAOS), colon carcinoma (Caco-2) and bladder carcinoma (SEG) (FIG. 4). We also identified control tumor cell lines HLA-A0201+Hsp70-(melanoma M113 and M44).

The breast cancer derived tumor cell line MCF-7 and the sarcoma derived tumor cell line SAOS showed the highest constitutive Hsp70 levels and were used in the recognition assay. Indeed, mCTL393 recognized specifically both the human HLA-A*0201+ Hsp70+tumor cell lines in a class-I specific manner as demonstrated by the TNF-α secretion assay (FIG. 5). This demonstrates particularly that HSP393 is an epitope correctly presented at the surface of Hsp70 over-expressing tumors and therefore that Hsp70 derived peptides represent good targets for CTLs in cancer immunotherapy.

The recognition of both HSP380 and HSP391 epitopes at the surface of tumor cells is also demonstrated.

### Example 5

### Activation of CTLs Specific for HSP Peptides by Peptide-Loaded Human Dendritic Cells

In order to investigate if human CTL can be induced against the three HSP epitopes previously selected, we stimulated CD8+ cells with peptide-loaded dendritic cells. We induced peptide specific CTL (see in FIG. 7A and FIG. 7B data for hCTL380, hCTL391 and hCTL393). These human CTL can recognize their specific epitope at the surface of a large panel of tumor cells of various histological types over-expressing Hsp70 described in FIG. 3.

These high affinity HSP70 polypeptide epitopes are further characterized by their ability to protect HHD mice from a challenge with the murine Hsp70 expressing EL-4/HHD tumor.

### Example 6

### Potency of the 22-mer p380 Polyepitope to Raise a Polyspecific Immune Response Against the three Hsp70 Epitopes

The 22 a.a. natural polyepitope was characterized for its ability to raise 3 distinct immune responses against the three HSP380, HSP391 and HSP393 epitopes in vivo in the HID murine model. HHD mice were vaccinated and their immune response against each epitope was assessed by ex vivo IFN-γ ELISPOT assay. Vaccination with the 22-mer polyepitope induced responses in most mice (FIG. 8A left panel and FIG. 8B). Interestingly, vaccination with the 22-mer polyepitope induced immune responses against all three Hsp70 epitopes, whereas vaccination with a mix of the three peptides gave rise to biased responses towards HSP391 only (FIG. 8A right panel). This demonstrates that the 22-mer polyepitope is advatageous to raise polyspecific immune responses against multiple Hsp70 epitopes in vivo in HHD mice.

We next investigated whether human dendritic cells pulsed with the 22-mer polyepitope were able to generate polyspecific CTL responses from human PBMC. Upon multiple restimulations of CD8+ cells with polyepitope-pulsed dendritic cells, CTL lines could be obtained which showed specificity towards all three Hsp70 epitopes (FIG. 9). This shows that the poleptiope is correctly processed from its 22-mer form into its three composing epitopes and that human dendritic cells pulsed with it are potent inducers of polyspecific immune responses against all three epitopes.

All together, these results demonstrate the potency of the 22-mer polyepitope to induce polyspecific immune responses against three Hsp70 epitopes both in vitro and in vivo.”

### REFERENCES

1. Rosenberg, S. A., J. C. Yang, D. J. Schwartzentruber, P. Hwu, F. M. Marincola, S. L. Topalian, N. P. Restifo, M. E. Dudley, S. L. Schwarz, P. J. Spiess, J. R. Wunderlich, M. R. Parkhurst, Y. Kawakami, C. A. Seipp, J. H. Einhorn, and D. E. White. 1998. Immunologic and therapeutic evaluation of a synthetic peptide vaccine for the treatment of patients with metastatic melanoma. Nat Med 4:321.
2. Nestle, F. O., S. Alijagic, M. Gilliet, Y. Sun, S. Grabbe, R. Dummer, G. Burg, and D. Schadendorf. 1998. Vaccination of melanoma patients with peptide- or tumor lysate-pulsed dendritic cells. Nat Med 4:328.
3. Marchand, M., N. van Baren, P. Weynants, V. Brichard, B. Dreno, M. H. Tessier, E. Rankin, G. Parmiani, F. Arienti, Y. Humblet, A. Bourlond, R. Vanwijck, D. Lienard, M. Beauduin, P. Y. Dietrich, V. Russo, J. Kerger, G. Masucci, E. Jager, J. De Greve, J. Atzpodien, F. Brasseur, P. G. Coulie, P. van der Bruggen, and T. Boon. 1999. Tumor regressions observed in patients with metastatic melanoma treated with an antigenic peptide encoded by gene MAGE-3 and presented by HLA-A1. Int J Cancer 80:219.
4. Kugler, A., G. Stuhler, P. Walden, G. Zoller, A. Zobywalski, P. Brossart, U. Trefzer, S. Ullrich, C. A. Muller, V. Becker, A. J. Gross, B. Hemmerlein, L. Kanz, G. A. Muller, and R. H. Ringert. 2000. Regression of human metastatic renal cell carcinoma after vaccination with tumor cell-dendritic cell hybrids. Nat Med 6:332.
5. Udono, H., and P. K Srivastava. 1993. Heat shock protein 70-associated peptides elicit specific cancer immunity. J Exp Med 178:1391.
6. Srivastava, P. K., A. Menoret, S. Basu, R. J. Binder, and K. L. McQuade. 1998. Heat shock proteins come of age: primitive functions acquire new roles in an adaptive world. Immunity 8:657.
7. Blachere, N. E., Z. Li, IL Y. Chandawarkar, R. Suto, N. S. Jaikaria, S. Basu, H. Udono, and P. K Srivastava. 1997. Heat shock protein-peptide complexes, reconstituted in vitro, elicit peptide-specific cytotoxic T lymphocyte response and tumor immunity. J Exp Med 186:1315.
8. Jaattela, M. 1999. Escaping cell death: survival proteins in cancer. Exp Cell Res 248:30.
9. Lazaris, A. C., E. B. Chatzigianni, D. Panoussopoulos, G. N. Tzimas, P. S. Davaris, and B. C. Golematis. 1997. Proliferating cell nuclear antigen and heat shock protein 70 immunolocalization in invasive ductal breast cancer not otherwise specified. Breast Cancer Res Treat 43:43.
10. Volm, M., J. Mattern, and G. Stammler. 1995. Up-regulation of heat shock protein 70 in adenocarcinomas of the lung in smokers. Anticancer Res 15:2607.
11. Trieb, K, T. Lechleitner, S. Lang, R. Windhager, R. Kotz, and S. Dirabofer. 1998. Heat shock protein 72 expression in osteosarcomas correlates with good response to neoadjuvant chemotherapy. Hum Pathol 29:1050.
12. Santarosa, M., D. Favaro, M. Quaia, and E. Galligioni. 1997. Expression of heat shock protein 72 in renal cell carcinoma: possible role and prognostic implications in cancer patients. Eur J Cancer 33:873.
13. Koshiyama, M., I. Konishi, M. Mandai, T. Komatsu, S. Yamamoto, K Nanbu, and T. Mori. 1995. Immunohistochemical analysis of p53 protein and 72 kDa heat shock protein (HSP72) expression in ovarian carcinomas. Correlation with clinicopathology and sex steroid receptor status. Virchows Arch 425:603.
14 Lazaris, A. C., Theodoropoulos, G. E., S. Davaris P. S., Panoussopoulos D., Nakopoulou L., Kittas C. and Golematis B. C. 1995. Heat shock protein 70 and HLA-DR molecules tissue expression. Prognostic implications in colorectal cancer. Dis Colon Rectum 38:739.
15 Park, C. S., I. S. Joo, S. Y. Song, D. S. Kim, D. S. Bae, and J. H. Lee. 1999. An immunohistochemical analysis of heat shock protein 70, p53, and estrogen receptor status in carcinoma of the uterine cervix. Gynecol Oncol 74:53.
16 Nanbu, K, I. Konishi, T. Komatsu, M. Mandai, S. Yamamoto, H. Kuroda, M. Koshiyama, and T. Mori. 1996. Expression of heat shock proteins HSP70 and HSP90 in endometrial carcinomas. Correlation with clinicopathology, sex steroid receptor status, and p53 protein expression. Cancer 77:330.
17. Jaattela, M., D. Wissing, K Kokholm, T. Kallunki, and M. Egeblad. 1998. Hsp70 exerts its anti-apoptotic function downstream of caspase-3-like proteases. Embo J 17:6124.
18. Nylandsted, J., K Brand, and M. Jaattela. 2000. Heat shock protein 70 is required for the survival of cancer cells. Ann N Y Acad Sci 926:122.
19. Nylandsted, J., M. Rohde, K Brand, L. Bastholm, F. Elling, and M. Jaattela. 2000. Selective depletion of heat shock protein 70 (Hsp70) activates a tumor-specific death program that is independent of caspases and bypasses Bcl-2. Proc Natl Acad Sci USA 97:7871.
20. Trieb, K., S. Lang, and R. Kotz. 2000. Heat-shock protein 72 in human osteosarcoma: T-lymphocyte reactivity and cytotoxicity. Pediatr Hematol Oncol 17:355.
21. Gaudin, C., F. Kremer, E. Angevin, V. Scott, and F. Triebel. 1999. A hsp70-2 mutation recognized by CTL on a human renal cell carcinoma J Immunol 162:1730.
22. Katoh, M., J. Koninkx, and U. Schumacher. 2000. Heat shock protein expression in human tumours grown in severe combined immunodeficient mice. Cancer Lett 161:113.
23. Johnsson, A., I. Zeelenberg, Y. Min, J. Hilinski, C. Berry, S. B. Howell, and G. Los. 2000. Identification of genes differentially expressed in association with acquired cisplatin resistance. Br J Cancer 83:1047.
24. Patel, B., A. Khaliq, J. Jarvis-Evans, M. Boulton, S. Arrol, M. Mackness, and D. McLeod. 1995. Hypoxia induces HSP 70 gene expression in human hepatoma (HEP G2) cells. Biochem Mol Biol Int 36:907.
25. Andersen, M. H., L. O. Pedersen, J. C. Becker, and P. T. Straten. 2001. Identification of a cytotoxic T lymphocyte response to the apoptosis inhibitor protein survivin in cancer patients. Cancer Res 61:869.
26. Espevik, T., and J. Nissen-Meyer. 1986. A highly sensitive cell line, WEHI 164 clone 13, for measuring cytotoxic factor/tumor necrosis factor from human monocytes. J Immunol Methods 95:99.
27. Tourdot, S., A. Scardino, E. Saloustrou, D. A. Gross, S. Pascolo, P. Cordopatis, F. A. Lemonnier, and K Kosmatopoulos. 2000. A general strategy to enhance immunogenicity of low-affinity HLA-A2. 1-associated peptides: implication in the identification of cryptic tumor epitopes. Eur J Immunol 30:3411.
28 Boyer A., Andreu G., Romet-Lemonne J. L., Fridman W. H. and Teillaud J. L. 1999. Generation of phagocytic MAK and MAC-DC for therapeutic use: characterization and in vitro functional properties. Exp. Hematol., 27, 751-761
29 Bocaccio C., Jacod S., Kaiser A., Boyer A., Abastado J. P. and Nardin A.

2002. Identification of a clinical-grade maturation factor for dendritic cells. J Immunother., 2002, 25(1) 88-96.
30 Keogh E. et al., 2001, The Journal of Immunology, 167: 787-796.

1. A peptide comprising an amino acid sequence of at least 8 contiguous amino acids, said amino acid sequence being at least 65% identical to a portion of the same length within the amino acid sequence of inducible Hsp70, characterized in that the peptide amino acid sequence differs from any amino acid sequence of the same length of the constitutive Hsc70 amino acid sequence by at least one amino acid, and that said peptide is able to induce in vitro or in vivo cytotoxic T lymphocytes able to specifically recognize cells naturally producing inducible Hsp70, said cytotoxic T lymphocytes being able to recognize both mutated and non mutated Hsp70 epitopes but not Hsc70 epitopes.

2. A peptide according to claim 1 wherein its amino acid sequence is at least 75% identical to a portion of the same length within the amino acid sequence of inducible Hsp70.

3. A peptide according to claim 1 wherein its amino acid sequence is identical to a portion of the same length within the amino acid sequence of inducible Hsp70.

4. A peptide according to claim 1 characterized in that its amino acid sequence differs from any amino acid sequence of the same length of the constitutive Hsc70 amino acid sequence by at least one amino acid located from the third to the last C-terminal amino acid of the peptide.

5. A peptide according to claim 1 characterized in that its amino acid sequence differs from any amino acid sequence of the same length of the constitutive Hsc70 amino acid sequence by at least one amino acid located from the fourth to the last C-terminal amino acid of the peptide.

6. A peptide according to claim 1 characterized in that its amino acid sequence comprises nine amino acids and differs from any amino acid sequence of the same length of the constitutive Hsc70 amino acid sequence by at least one amino acid located from the fourth to the ninth amino acid of the peptide.

7. A peptide according to claim 1 characterized in that its amino acid sequence comprises ten amino acids and differs from any amino acid sequence of the same length of the constitutive Hsc70 amino acid sequence by at least one amino acid located from the fourth to the tenth amino acid of the peptide.

8. A peptide according to claim 1 characterized in that its amino acid sequence differs from any amino acid sequence of the same length of the inducible Hsp70 amino acid sequence by at least one amino acid located at position 1, 2 or last C-terminal of the peptide.

9. A peptide according to claim 1 characterized in that its amino acid sequence from the third amino acid to the one before the last C-terminal amino acid is identical to a portion of the same length within the amino acid sequence of inducible Hsp70.

10. A peptide according to claim 1 characterized in that its amino acid sequence differs from any amino acid sequence of the same length of the inducible Hsp70 amino acid sequence by at least one amino acid.

11. A peptide according to claim 1 comprising or consisting of any amino acid sequence having at least 65% homology with, or identical to, any member of the group of amino acid sequence consisting of:

| | |
|---|---|
| QVALNPQNTV, | (SEQ ID NO: 1) |
| LIGRKFGDPV, | (SEQ ID NO: 2) |
| DMKHWPFQVI, | (SEQ ID NO: 3) |
| DMKHWPFQV, | (SEQ ID NO: 4) |
| VINDGDKPKV, | (SEQ ID NO: 5) |
| EISSMVLTKM, | (SEQ ID NO: 6) |
| YLGYPVTNAV, | (SEQ ID NO: 7) |
| PVTNAVITV, | (SEQ ID NO: 8) |
| GVIAGLNVL, | (SEQ ID NO: 9) |
| VIAGLNVLRI, | (SEQ ID NO: 10) |
| SILTIDDGI, | (SEQ ID NO: 11) |
| TIDDGIFEV, | (SEQ ID NO: 12) |
| DISQNKRAV, | (SEQ ID NO: 13) |
| TLSSSTQASL, | (SEQ ID NO: 14) |
| EIDSLFEGI, | (SEQ ID NO: 15) |
| DLFRSTLEPV, | (SEQ ID NO: 16) |
| TLEPVEKAL, | (SEQ ID NO: 17) |
| KLDKAQIHDL, | (SEQ ID NO: 18) |
| LVLVGGSTRI, | (SEQ ID NO: 19) |
| RIPKVQKLL, | (SEQ ID NO: 20) |
| ILMGDKSENV, | (SEQ ID NO: 21) |
| LMGDKSENV, | (SEQ ID NO: 22) |
| DLLLLDVAPL, | (SEQ ID NO: 23) |
| LLLLDVAPL, | (SEQ ID NO: 24) |
| LLLDVAPLSL, | (SEQ ID NO: 25) |
| LLDVAPLSL, | (SEQ ID NO: 26) |
| DVAPLSLGL, | (SEQ ID NO: 27) |
| SLGLETAGGV, | (SEQ ID NO: 28) |
| GLETAGGVM, | (SEQ ID NO: 29) |
| ALIKRNSTI, | (SEQ ID NO: 30) |
| TIPTKQTQI, | (SEQ ID NO: 31) |
| ALESYAFNM, | (SEQ ID NO: 32) |
| LLGRFELSGI, | (SEQ ID NO: 33) |
| RLSKEEIERM, | (SEQ ID NO: 34) |
| KISEADKKKV, | (SEQ ID NO: 35) |
| KVLDKCQEV, | (SEQ ID NO: 36) |
| KVLDKCQEVI, | (SEQ ID NO: 37) |
| VLDKCQEVI, | (SEQ ID NO: 38) |
| VISWLDANTL, | (SEQ ID NO: 39) |
| ELEQVCNPI, | (SEQ ID NO: 40) |
| ELEQVCNPII, | (SEQ ID NO: 41) |
| QVCNPIISGL. | (SEQ ID NO: 42) |

12. A peptide according to claim 1 comprising or consisting of any amino acid sequence having at least 65% homology with, or identical to, any member of the group of amino acid sequence consisting of:

| | |
|---|---|
| QVALNPQNTV, | (SEQ ID NO: 1) |
| LIGRKFGDPV, | (SEQ ID NO: 2) |
| DMKHQPFQVI, | (SEQ ID NO: 3) |
| DMKHQPFQV, | (SEQ ID NO: 4) |
| VINDGDKPKV, | (SEQ ID NO: 5) |
| YLGYPVTNAV, | (SEQ ID NO: 7) |
| PVTNAVITV, | (SEQ ID NO: 8) |
| SILTIDDGI, | (SEQ ID NO: 11) |
| DISQNKRAV, | (SEQ ID NO: 13) |
| TLSSSTQASAL, | (SEQ ID NO: 14) |
| EIDSLFEGI, | (SEQ ID NO: 15) |
| DLFRSTLEPV, | (SEQ ID NO: 16) |
| KLDKAQIHDL, | (SEQ ID NO: 18) |
| RIPKVQKLL, | (SEQ ID NO: 20) |
| DLLLLDVAPL, | (SEQ ID NO: 23) |
| LLLLDVAPL, | (SEQ ID NO: 24) |
| LLLDVAPLSL, | (SEQ ID NO: 25) |
| LLDVAPLSL, | (SEQ ID NO: 26) |
| DVAPLSLGL, | (SEQ ID NO: 27) |
| SLGLETAGGV, | (SEQ ID NO: 28) |
| ALIKRNSTI, | (SEQ ID NO: 30) |
| TIPTKQTQI, | (SEQ ID NO: 31) |
| LLGRFELSGI, | (SEQ ID NO: 33) |
| RLSKEEIERM, | (SEQ ID NO: 34) |
| KISEADKKKV, | (SEQ ID NO: 35) |
| KVLDKCQEV, | (SEQ ID NO: 36) |
| KVLDKCQEVI, | (SEQ ID NO: 37) |
| VLDKCQEVI, | (SEQ ID NO: 38) |
| VISWLDANTL, | (SEQ ID NO: 39) |
| ELEQVCNPI, | (SEQ ID NO: 40) |
| ELEQVCNPII, | (SEQ ID NO: 41) |
| QVCNPIISGL. | (SEQ ID NO: 42) |

13. A peptide according to claim 1 comprising or consisting of any amino acid sequence having at least 65% homology with, or identical to, any member of the group of amino acid sequence consisting of:

| | |
|---|---|
| ILNVTATDK, | (SEQ ID NO: 43) |
| SLFEGIDFY, | (SEQ ID NO: 44) |
| ALNPQNTVF, | (SEQ ID NO: 45) |
| ILTIDDGIF, | (SEQ ID NO: 46) |
| QVSYKGETK, | (SEQ ID NO: 47) |
| AVITVPAYF, | (SEQ ID NO: 48) |
| LVNHFVEEF, | (SEQ ID NO: 49) |
| KVQKLLQDF, | (SEQ ID NO: 50) |
| ALESYAFNMK, | (SEQ ID NO: 51) |
| WLDANTLAEK, | (SEQ ID NO: 52) |
| RLVNHFVEEF, | (SEQ ID NO: 53) |
| QVINDGDKPK, | (SEQ ID NO: 54) |
| LVNHFVEEFK, | (SEQ ID NO: 55) |
| FVEEFKRKHK, | (SEQ ID NO: 56) |
| EVQRERVSAK, | (SEQ ID NO: 57) |
| AVEDEGLKGK, | (SEQ ID NO: 58) |
| KVQKLLQDFF | (SEQ ID NO: 59) |

14. A peptide according to claim 1 comprising or consisting of any amino acid sequence having at least 65% homology with, or identical to, any member of the group of amino acid sequence consisting of:

| | |
|---|---|
| NPQNTVFDA, | (SEQ ID NO: 60) |
| NPQNTVFDAK, | (SEQ ID NO: 61) |
| DPVVQSDMK, | (SEQ ID NO: 62) |
| DPVVQSDMKH, | (SEQ ID NO: 63) |
| WPFQVINDG, | (SEQ ID NO: 64) |
| WPFQVINDGD, | (SEQ ID NO: 65) |
| KPKVQVSYK, | (SEQ ID NO: 66) |
| KPKVQVSYKG, | (SEQ ID NO: 67) |
| YPEEISSMV, | (SEQ ID NO: 68) |
| YPEEISSMVL, | (SEQ ID NO: 69) |
| YPVTNAVIT, | (SEQ ID NO: 70) |
| YPVTNAVITV, | (SEQ ID NO: 71) |
| EPVEKALRD, | (SEQ ID NO: 72) |
| EPVEKALRDA, | (SEQ ID NO: 73) |
| IPKVQKLLQ, | (SEQ ID NO: 74) |
| IPKVQKLLQD, | (SEQ ID NO: 75) |
| APLSLGLET, | (SEQ ID NO: 76) |
| APLSLGLETA, | (SEQ ID NO: 77) |
| IPTKQTQIF, | (SEQ ID NO: 78) |
| IPTKQTQIFT, | (SEQ ID NO: 79) |
| NPIISGLYQ, | (SEQ ID NO: 80) |
| NPIISGLYQG, | (SEQ ID NO: 81) |
| GPGPGGFGA, | (SEQ ID NO: 82) |
| GPGPGGFGAQ, | (SEQ ID NO: 83) |
| GPGGFGAQG, | (SEQ ID NO: 84) |
| GPGGFGAQGP, | (SEQ ID NO: 85) |
| GPKGGSGSG, | (SEQ ID NO: 86) |
| GPKGGSGSGP | (SEQ ID NO: 87) |

15. A polyepitope comprising a peptide according to claim 1 and at least a second amino acid sequence selected from the group consisting of:
An amino acid sequence identical to said first amino acid sequence,
An amino acid sequence chosen from:

| | |
|---|---|
| QVALNPQNTV, | (SEQ ID NO: 1) |
| LIGRKFGDPV, | (SEQ ID NO: 2) |
| DMKHQPFQVI, | (SEQ ID NO: 3) |
| DMKHWPFQV, | (SEQ ID NO: 4) |
| VINDGDKPKV, | (SEQ ID NO: 5) |
| EISSMVLTKM, | (SEQ ID NO: 6) |
| YLGYPVTNAV, | (SEQ ID NO: 7) |
| PVTNAVITV, | (SEQ ID NO: 8) |
| GVIAGLNVL, | (SEQ ID NO: 9) |
| VIAGLNVLRI, | (SEQ ID NO: 10) |
| SILTIDDGI, | (SEQ ID NO: 11) |
| TIDDGIFEV, | (SEQ ID NO: 12) |
| DISQNKRAV, | (SEQ ID NO: 13) |
| TLSSSTQASL, | (SEQ ID NO: 14) |
| EIDSLFEGI, | (SEQ ID NO: 15) |
| DLFRSTLEPV, | (SEQ ID NO: 16) |
| TLEPVEKAL, | (SEQ ID NO: 17) |
| KLDKAQIHDL, | (SEQ ID NO: 18) |
| LVLVGGSTRI, | (SEQ ID NO: 19) |
| RIPKVQKLL, | (SEQ ID NO: 20) |
| ILMGDKSENV, | (SEQ ID NO: 21) |
| LMGDKSENV, | (SEQ ID NO: 22) |
| DLLLLDVAPL, | (SEQ ID NO: 23) |
| LLLLDVAPL, | (SEQ ID NO: 24) |
| LLLDVAPLSL, | (SEQ ID NO: 25) |
| LLDVAPLSL, | (SEQ ID NO: 26) |
| DVAPLSLGL, | (SEQ ID NO: 27) |
| SLGLETAGGV, | (SEQ ID NO: 28) |
| GLETAGGVM, | (SEQ ID NO: 29) |
| ALIKRNSTI, | (SEQ ID NO: 30) |
| TIPTKQTQI, | (SEQ ID NO: 31) |
| ALESYAFNM, | (SEQ ID NO: 32) |
| LLGRFELSGI, | (SEQ ID NO: 33) |
| RLSKEEIERM, | (SEQ ID NO: 34) |
| KISEADDKKKV, | (SEQ ID NO: 35) |
| KVLDKCQEV, | (SEQ ID NO: 36) |
| KVLDKCQEVI, | (SEQ ID NO: 37) |
| VLDKCQEVI, | (SEQ ID NO: 38) |
| VISWLDANTL, | (SEQ ID NO: 39) |
| ELEQVCNPI, | (SEQ ID NO: 40) |
| ELEQVCNPII, | (SEQ ID NO: 41) |
| QVCNPIISGL. | (SEQ ID NO: 42) |

16. A polyepitope according to claim 15 comprising the amino acid sequence LMGDKSENVQDLLLLDVAPLSL (SEQ ID NO: 88).

17. A peptide according to claim 1 characterized in that it is linked to a carrier which can be a biodegradable microparticle

18. A nucleic acid sequence encoding for a peptide according to claim 1.

19. A nucleic acid sequence according to claim 18 characterized in that it is linked to a vector.

20. A method for inducing in vitro a cytotoxic T lymphocyte response that specifically targets cells naturally expressing inducible Hsp70, administering a peptide according to claim 1.

21. A cytotoxic T lymphocyte such as obtained by a method according to claim 20.

22. An antigen presenting cell presenting on its surface at least one peptide according to claim 1.

23. A pharmaceutical composition or a vaccine comprising at least, in association with a pharmaceutically acceptable vehicle, a peptide according claim 1.

24. A pharmaceutical composition or a vaccine comprising at least, in association with a pharmaceutically acceptable vehicle, a peptide comprising an amino acid sequence selected from the group consisting of: SLFEGIDFY (SEQ ID NO: 44), SLFEGIDFYT (SEQ ID NO: 89), and LMGDKSENV (SEQ ID NO: 22).

25. A pharmaceutical composition or a vaccine comprising at least, in association with a pharmaceutically acceptable vehicle, a peptide comprising or consisting of an amino acid sequence being at least 65% identical to the following sequence LMGDKSENV (SEQ ID NO: 22).

26. A method for preparing a drug for the treatment of cancer or a viral disease in a subject, comprising administering to said subject a peptide according to claim 1.

27. The method according to claim 26, wherein the peptide has an amino acid sequence selected from the group consisting of: SLFEGIDFY (SEQ ID NO: 44), SLFEGIDFYT (SEQ ID NO: 89), LMGDKSENV (SEQ ID NO: 22 for the preparation of a drug useful in the treatment of cancer.

28. A method for preparing a drug for the treatment of cancer or a viral disease in a subject, comprising administering to said subject an amino acid sequence of LMGDKSENV (SEQ ID NO: 22).


SEQUENCE LISTING (190 sequences)

SEQ ID NO: 1 - protein, 10 aa, Homo sapiens
QVALNPQNTV

SEQ ID NO: 2 - protein, 10 aa, Homo sapiens
LIGRKFGDPV

SEQ ID NO: 3 - protein, 10 aa, Homo sapiens
DMKHWPFQVI

SEQ ID NO: 4 - protein, 9 aa, Homo sapiens
DMKHWPFQV

SEQ ID NO: 5 - protein, 10 aa, Homo sapiens
VINDGDKPKV

SEQ ID NO: 6 - protein, 10 aa, Homo sapiens
EISSMVLTKM

SEQ ID NO: 7 - protein, 10 aa, Homo sapiens
YLGYPVTNAV

SEQ ID NO: 8 - protein, 9 aa, Homo sapiens
PVTNAVITV

SEQ ID NO: 9 - protein, 9 aa, Homo sapiens
GVIAGLNVL

SEQ ID NO: 10 - protein, 10 aa, Homo sapiens
VIAGLNVLRI

SEQ ID NO: 11 - protein, 9 aa, Homo sapiens
SILTIDDGI

SEQ ID NO: 12 - protein, 9 aa, Homo sapiens
TIDDGIFEV

SEQ ID NO: 13 - protein, 9 aa, Homo sapiens
DISQNKRAV

SEQ ID NO: 14 - protein, 10 aa, Homo sapiens
TLSSSTQASL

SEQ ID NO: 15 - protein, 9 aa, Homo sapiens
EIDSLFEGI

SEQ ID NO: 16 - protein, 10 aa, Homo sapiens
DLFRSTLEPV

SEQ ID NO: 17 - protein, 9 aa, Homo sapiens
TLEPVEKAL

SEQ ID NO: 18 - protein, 10 aa, Homo sapiens
KLDKAQIHDL

SEQ ID NO: 19 - protein, 10 aa, Homo sapiens
LVLVGGSTRI

SEQ ID NO: 20 - protein, 9 aa, Homo sapiens
RIPKVQKLL

SEQ ID NO: 21 - protein, 10 aa, Homo sapiens
ILMGDKSENV

SEQ ID NO: 22 - protein, 9 aa, Homo sapiens
LMGDKSENV

SEQ ID NO: 23 - protein, 10 aa, Homo sapiens
DLLLLDVAPL

SEQ ID NO: 24 - protein, 9 aa, Homo sapiens
LLLLDVAPL

SEQ ID NO: 25 - protein, 10 aa, Homo sapiens
LLLDVAPLSL

SEQ ID NO: 26 - protein, 9 aa, Homo sapiens
LLDVAPLSL

SEQ ID NO: 27 - protein, 9 aa, Homo sapiens
DVAPLSLGL

SEQ ID NO: 28 - protein, 10 aa, Homo sapiens
SLGLETAGGV

SEQ ID NO: 29 - protein, 9 aa, Homo sapiens
GLETAGGVM

SEQ ID NO: 30 - protein, 9 aa, Homo sapiens
ALIKRNSTI

SEQ ID NO: 31 - protein, 9 aa, Homo sapiens
TIPTKQTQI

SEQ ID NO: 32 - protein, 9 aa, Homo sapiens
ALESYAFNM

SEQ ID NO: 33 - protein, 10 aa, Homo sapiens
LLGRFELSGI

SEQ ID NO: 34 - protein, 10 aa, Homo sapiens
RLSKEEIERM

SEQ ID NO: 35 - protein, 10 aa, Homo sapiens
KISEADKKKV

SEQ ID NO: 36 - protein, 9 aa, Homo sapiens
KVLDKCQEV

SEQ ID NO: 37 - protein, 10 aa, Homo sapiens
KVLDKCQEVI

SEQ ID NO: 38 - protein, 9 aa, Homo sapiens
VLDKCQEVI

SEQ ID NO: 39 - protein, 10 aa, Homo sapiens
VISWLDANTL

SEQ ID NO: 40 - protein, 9 aa, Homo sapiens
ELEQVCNPI

SEQ ID NO: 41 - protein, 10 aa, Homo sapiens
ELEQVCNPII

SEQ ID NO: 42 - protein, 10 aa, Homo sapiens
QVCNPIISGL

SEQ ID NO: 43 - protein, 9 aa, Homo sapiens
ILNVTATDK

SEQ ID NO: 44 - protein, 9 aa, Homo sapiens
SLFEGIDFY

SEQ ID NO: 45 - protein, 9 aa, Homo sapiens
ALNPQNTVF

SEQ ID NO: 46 - protein, 9 aa, Homo sapiens
ILTIDDGIF

SEQ ID NO: 47 - protein, 9 aa, Homo sapiens
QVSYKGETK

SEQ ID NO: 48 - protein, 9 aa, Homo sapiens
AVITVPAYF

SEQ ID NO: 49 - protein, 9 aa, Homo sapiens
LVNHFVEEF

SEQ ID NO: 50 - protein, 9 aa, Homo sapiens
KVQKLLQDF

SEQ ID NO: 51 - protein, 10 aa, Homo sapiens
ALESYAFNMK

SEQ ID NO: 52 - protein, 10 aa, Homo sapiens
WLDANTLAEK

SEQ ID NO: 53 - protein, 10 aa, Homo sapiens
RLVNHFVEEF

SEQ ID NO: 54 - protein, 10 aa, Homo sapiens
QVINDGDKPK

SEQ ID NO: 55 - protein, 10 aa, Homo sapiens
LVNHFVEEFK

SEQ ID NO: 56 - protein, 10 aa, Homo sapiens
FVEEFKRKHK

SEQ ID NO: 57 - protein, 10 aa, Homo sapiens
EVQRERVSAK

SEQ ID NO: 58 - protein, 10 aa, Homo sapiens
AVEDEGLKGK

SEQ ID NO: 59 - protein, 10 aa, Homo sapiens
KVQKLLQDFF

SEQ ID NO: 60 - protein, 9 aa, Homo sapiens
NPQNTVFDA

SEQ ID NO: 61 - protein, 10 aa, Homo sapiens
NPQNTVFDAK

SEQ ID NO: 62 - protein, 9 aa, Homo sapiens
DPVVQSDMK

SEQ ID NO: 63 - protein, 10 aa, Homo sapiens
DPVVQSDMKH

SEQ ID NO: 64 - protein, 9 aa, Homo sapiens
WPFQVINDG

SEQ ID NO: 65 - protein, 10 aa, Homo sapiens
WPFQVINDGD

SEQ ID NO: 66 - protein, 9 aa, Homo sapiens
KPKVQVSYK

SEQ ID NO: 67 - protein, 10 aa, Homo sapiens
KPKVQVSYKG

SEQ ID NO: 68 - protein, 9 aa, Homo sapiens
YPEEISSMV

SEQ ID NO: 69 - protein, 10 aa, Homo sapiens
YPEEISSMVL

SEQ ID NO: 70 - protein, 9 aa, Homo sapiens
YPVTNAVIT

SEQ ID NO: 71 - protein, 10 aa, Homo sapiens
YPVTNAVITV

SEQ ID NO: 72 - protein, 9 aa, Homo sapiens
EPVEKALRD

SEQ ID NO: 73 - protein, 10 aa, Homo sapiens
EPVEKALRDA

SEQ ID NO: 74 - protein, 9 aa, Homo sapiens
IPKVQKLLQ

SEQ ID NO: 75 - protein, 10 aa, Homo sapiens
IPKVQKLLQD

SEQ ID NO: 76 - protein, 9 aa, Homo sapiens
APLSLGLET

SEQ ID NO: 77 - protein, 10 aa, Homo sapiens
APLSLGLETA

SEQ ID NO: 78 - protein, 9 aa, Homo sapiens
IPTKQTQIF

SEQ ID NO: 79 - protein, 10 aa, Homo sapiens
IPTKQTQIFT

SEQ ID NO: 80 - protein, 9 aa, Homo sapiens
NPIISGLYQ

SEQ ID NO: 81 - protein, 10 aa, Homo sapiens
NPIISGLYQG

SEQ ID NO: 82 - protein, 9 aa, Homo sapiens
GPGPGGFGA

SEQ ID NO: 83 - protein, 10 aa, Homo sapiens
GPGPGGFGAQ

SEQ ID NO: 84 - protein, 9 aa, Homo sapiens
GPGGFGAQG

SEQ ID NO: 85 - protein, 10 aa, Homo sapiens
GPGGFGAQGP

SEQ ID NO: 86 - protein, 9 aa, Homo sapiens
GPKGGSGSG

SEQ ID NO: 87 - protein, 10 aa, Homo sapiens
GPKGGSGSGP

SEQ ID NO: 88 - protein, 22 aa, Homo sapiens
LMGDKSENVQDLLLLDVAPLSL

SEQ ID NO: 89 - protein, 10 aa, Homo sapiens
SLFEGIDFYT

SEQ ID NO: 90 - protein, 9 aa, Homo sapiens
IVGAETFYV

SEQ ID NO: 91 - protein, 13 aa, Homo sapiens
TPPAYRPPNAPIL

SEQ ID NO: 92 - protein, 9 aa, Homo sapiens
NPTNTVFDA

SEQ ID NO: 93 - protein, 10 aa, Homo sapiens
NPTNTVFDAK

SEQ ID NO: 94 - protein, 9 aa, Homo sapiens
DAVVQSDMK

SEQ ID NO: 95 - protein, 10 aa, Homo sapiens
DAVVQSDMKH

SEQ ID NO: 96 - protein, 9 aa, Homo sapiens
WPFMVVNDA

SEQ ID NO: 97 - protein, 10 aa, Homo sapiens
WPFMVVNDAG

SEQ ID NO: 98 - protein, 9 aa, Homo sapiens
RPKVQVEYK

SEQ ID NO: 99 - protein, 10 aa, Homo sapiens
RPKVQVEYKG

SEQ ID NO: 100 - protein, 9 aa, Homo sapiens
YPEEVSSMV

SEQ ID NO: 101 - protein, 10 aa, Homo sapiens
YPEEVSSMVL

SEQ ID NO: 102 - protein, 9 aa, Homo sapiens
KTVTNAVVT

SEQ ID NO: 103 - protein, 10 aa, Homo sapiens
KTVTNAVVTV

SEQ ID NO: 104 - protein, 10 aa, Homo sapiens
DPVEKALRDA

SEQ ID NO: 105 - protein, 9 aa, Homo sapiens
IPKIQKLLQ

SEQ ID NO: 106 - protein, 10 aa, Homo sapiens
IPKIQKLLQD

SEQ ID NO: 107 - protein, 9 aa, Homo sapiens
TPLSLGIET

SEQ ID NO: 108 - protein, 10 aa, Homo sapiens
TPLSLGIETA

SEQ ID NO: 109 - protein, 9 aa, Homo sapiens
IPTKQTQTF

SEQ ID NO: 110 - protein, 10 aa, Homo sapiens
IPTKQTQTFT

SEQ ID NO: 111 - protein, 9 aa, Homo sapiens
NPIITKLYQ

SEQ ID NO: 112 - protein, 10 aa, Homo sapiens
NPIITKLYQS

SEQ ID NO: 113 - protein, 9 aa, Homo sapiens
GGMPGGFPG

SEQ ID NO: 114 - protein, 10 aa, Homo sapiens
GGMPGGFPGG

SEQ ID NO: 115 - protein, 9 aa, Homo sapiens
MPGGFPGGG

SEQ ID NO: 116 - protein, 10 aa, Homo sapiens
MPGGFPGGGA

SEQ ID NO: 117 - protein, 9 aa, Homo sapiens
PPSGGASSG

SEQ ID NO: 118 - protein, 10 aa, Homo sapiens
PPSGGASSGP

SEQ ID NO: 119 - protein, 10 aa, Homo sapiens
QVAMNPTNTV

SEQ ID NO: 120 - protein, 10 aa, Homo sapiens
LIGRRFDDAV

SEQ ID NO: 121 - protein, 10 aa, Homo sapiens
DMKHWPFMVV

SEQ ID NO: 122 - protein, 9 aa, Homo sapiens
DMKHWPFMV

SEQ ID NO: 123 - protein, 10 aa, Homo sapiens
VVNDAGRPKV

SEQ ID NO: 124 - protein, 10 aa, Homo sapiens
YLGKTVTNAV

SEQ ID NO: 125 - protein, 9 aa, Homo sapiens
TVTNAVVTV

SEQ ID NO: 126 - protein, 9 aa, Homo sapiens
SILTIEDGI

SEQ ID NO: 127 - protein, 9 aa, Homo sapiens
DISENKRAV

SEQ ID NO: 128 - protein, 10 aa, Homo sapiens
TLSSSTQASI

SEQ ID NO: 129 - protein, 9 aa, Homo sapiens
EIDSLYEGI

SEQ ID NO: 130 - protein, 10 aa, Homo sapiens
DLFRGTLDPV

SEQ ID NO: 131 - protein, 10 aa, Homo sapiens
KLDKSQIHDI

SEQ ID NO: 132 - protein, 9 aa, Homo sapiens
RIPKIQKLL

SEQ ID NO: 133 - protein, 10 aa, Homo sapiens
DLLLLDVTPL

SEQ ID NO: 134 - protein, 9 aa, Homo sapiens
LLLLDVTPL

SEQ ID NO: 135 - protein, 10 aa, Homo sapiens
LLLDVTPLSL

SEQ ID NO: 136 - protein, 9 aa, Homo sapiens
LLDVTPLSL

SEQ ID NO: 137 - protein, 9 aa, Homo sapiens
DVTPLSLGI

SEQ ID NO: 138 - protein, 10 aa, Homo sapiens
SLGIETAGGV

SEQ ID NO: 139 - protein, 9 aa, Homo sapiens
VLIKRNTTI

SEQ ID NO: 140 - protein, 9 aa, Homo sapiens
TIPTKQTQT

SEQ ID NO: 141 - protein, 10 aa, Homo sapiens
LLGKFELTGI

SEQ ID NO: 142 - protein, 10 aa, Homo sapiens
RLSKEDIERM

SEQ ID NO: 143 - protein, 10 aa, Homo sapiens
KINDEDKQKI

SEQ ID NO: 144 - protein, 9 aa, Homo sapiens
KILDKCNEI

SEQ ID NO: 145 - protein, 10 aa, Homo sapiens
KILDKCNEII

SEQ ID NO: 146 - protein, 9 aa, Homo sapiens
ILDKCNEII

SEQ ID NO: 147 - protein, 10 aa, Homo sapiens
IINWLDKNQT

SEQ ID NO: 148 - protein, 9 aa, Homo sapiens
ELEKVCNPI

SEQ ID NO: 149 - protein, 10 aa, Homo sapiens
ELEKVCNPII

SEQ ID NO: 150 - protein, 10 aa, Homo sapiens
KVCNPIITKL

SEQ ID NO: 151 - protein, 9 aa, Homo sapiens
ILNVSAVDK

SEQ ID NO: 152 - protein, 9 aa, Homo sapiens
AMNPTNTVF

SEQ ID NO: 153 - protein, 9 aa, Homo sapiens
ILTIEDGIF

SEQ ID NO: 154 - protein, 9 aa, Homo sapiens
MVNHFIAEF

SEQ ID NO: 155 - protein, 10 aa, Homo sapiens
WLDKNQTAEK

SEQ ID NO: 156 - protein, 10 aa, Homo sapiens
RMVNHFIAEF

SEQ ID NO: 157 - protein, 10 aa, Homo sapiens
MVVNDAGRPK

SEQ ID NO: 158 - protein, 10 aa, Homo sapiens
MVNHFIAEFK

SEQ ID NO: 159 - protein, 10 aa, Homo sapiens
EKQRDKVSSK

SEQ ID NO: 160 - protein, 10 aa, Homo sapiens
TVEDEKLQGK

SEQ ID NO: 161 - protein, 9 aa, Homo sapiens
WPFMVVNDA

SEQ ID NO: 162 - protein, 10 aa, Homo sapiens
WPFMVVNDAG

SEQ ID NO: 163 - protein, 9 aa, Homo sapiens
RPKVQVEYK

SEQ ID NO: 164 - protein, 10 aa, Homo sapiens
RPKVQVEYKG

SEQ ID NO: 165 - protein, 9 aa, Homo sapiens
YPEEVSSMV

SEQ ID NO: 166 - protein, 10 aa, Homo sapiens
YPEEVSSMVL

SEQ ID NO: 167 - protein, 9 aa, Homo sapiens
KTVTNAVVT

SEQ ID NO: 168 - protein, 10 aa, Homo sapiens
KTVTNAVVTV

SEQ ID NO: 169 - protein, 9 aa, Homo sapiens
IPKIQKLLQ

SEQ ID NO: 170 - protein, 10 aa, Homo sapiens
IPKIQKLLQD

SEQ ID NO: 171 - protein, 9 aa, Homo sapiens
TPLSLGIET

SEQ ID NO: 172 - protein, 10 aa, Homo sapiens
TPLSLGIETA

SEQ ID NO: 173 - protein, 9 aa, Homo sapiens
IPTKQTQTF

SEQ ID NO: 174 - protein, 10 aa, Homo sapiens
IPTKQTQTFT

SEQ ID NO: 175 - protein, 9 aa, Homo sapiens
NPIITKLYQ

SEQ ID NO: 176 - protein, 10 aa, Homo sapiens
NPIITKLYQS

SEQ ID NO: 177 - protein, 9 aa, Homo sapiens
GGMPGGFPG

SEQ ID NO: 178 - protein, 10 aa, Homo sapiens
GGMPGGFPGG

SEQ ID NO: 179 - protein, 9 aa, Homo sapiens
MPGGFPGGG

SEQ ID NO: 180 - protein, 10 aa, Homo sapiens
MPGGFPGGGA

SEQ ID NO: 181 - protein, 9 aa, Homo sapiens
PPSGGASSG

SEQ ID NO: 182 - protein, 10 aa, Homo sapiens
PPSGGASSGP

SEQ ID NO: 183 - protein, 9 aa, Homo sapiens
YLGYPVTNA

SEQ ID NO: 184 - protein, 9 aa, Homo sapiens
YLGKTVTNA

SEQ ID NO: 185 - protein, 10 aa, Homo sapiens
SLYEGIDFYT

SEQ ID NO: 186 - protein, 9 aa, Homo sapiens
LSGDKSENV

SEQ ID NO: 187 - protein, 10 aa, Homo sapiens
STRIPKVQKL

SEQ ID NO: 188 - protein, 10 aa, Homo sapiens
STRIPKIQKL

SEQ ID NO: 189 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 190 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD